(19) United States
(12) Patent Application Publication
(10) Pub. No.: US 2006/0142171 A1
(43) Pub. Date: Jun. 29, 2006

(54) Novel alkaline protease

(76) Inventors: Rajni Hatti-Kaul, Lund (SE); Bo Mattiasson, Hjarup (SE); Amare Gessesse, Norresundby (DK)

Correspondence Address: GAUTHIER & CONNORS, LLP, 225 FRANKLIN STREET, BOSTON, MA, 02110, US

(21) Appl. No.: 11/235,479

(22) Filed: Sep. 26, 2005

Related U.S. Application Data
(63) Continuation of application No. PCT/SE04/00405, filed on Mar. 19, 2004.

(30) Foreign Application Priority Data
Mar. 24, 2003 (SE) 0300778-8
Nov. 25, 2003 (SE) 0303120-0

(51) Int. Cl. C11D 3/386; C07H 21/04; C12P 21/06; C12N 9/16; C12N 15/74

(52) U.S. Cl. 510/320; 435/69.1; 435/196; 435/252.3; 435/320.1; 536/23.2

(57) ABSTRACT

The present invention relates to a novel isolated and purified alkaline protease from Nesterenkonia sp. nov. strain, having the following characteristics: molecular weight of 23 kilodalton, melting temperature of about 74° C. at pH 7-10, calcium independent for activity and stability, maximal protease activity at pH 10, and being a serine protease, as well as a composition comprising the enzyme, method for producing the enzyme, method of degrading proteinaceous material, an isolated strain Nesterenkonia sp. nov..

### TECHNICAL FIELD

The present invention relates to a novel alkaline protease derived from an isolated Nesterenkonia sp., the isolated strain of novel Nesterenkonia sp., compositions comprising such an alkaline protease, method of producing the alkaline protease, method for degrading proteinaceous material, as well as method for culturing the novel strain.

Proteases constitute one the most important group of industrial enzymes. Many distinct families of proteases exist of which alkaline proteases are one of the largest and commercially important (Siezen and Leunissen 1997). Alkaline proteases are produced by a wide variety of bacteria, yeasts and moulds and are also found in mammalian tissues (Anwar and Saleemuddin 1998). Among various alkaline proteases, bacterial alkaline proteases have been extensively studied in view of their structural-function relationship and catalytic mechanism, these enzymes attracted considerable interest, for their industrial application in peptide synthesis and resolution of the mixture of D,L-amino acids, protein-degrading additives in detergents, leather processing, silver recovery, and food processing industry (Banerjee et al. 1999, Gupta et al. 1999, Horikoshi 1999, Kalisz 1988, Kumar and Takagi 1999).

Serine proteases constitute among the most important group of industrial enzymes; they have a widespread occurrence and exist in many distinct families. Alkaline proteases of the subtilisin family produced by Bacillus species form the largest subgroup of serine proteases (Siezen et al., 1991). These enzymes have become commercially important as protein degrading component of washing detergents, the most suitable being the proteases from alkaliphilic bacilli (Banerjee et al., 1999; Kalisz, 1988; Outtrup & Boyce, 1990). Subtilisin-like proteases are also produced by other bacteria, archaea, fungi, yeasts, and even higher eukaryotes (Anwar & Saleemuddin, 1998).

They can also be used to convert fibrous proteins (e.g. horn, feather, nails and hair) that are abundantly available in nature as wastes to useful biomass, protein concentrate or amino acids (Anwar and Saleemuddin 1998). For these applications, as well as to design inhibitors, knowledge of the substrate specificity is absolutely necessary.

The important feature of alkaline proteases is their ability to discriminate among competing substrates and utility of these enzymes depend often on their substrate specificity. The common feature present in the structure of alkaline proteases is that the enzymes possess an identical catalytic triad of Ser, His, and Asp residues and a α/β fold (Siezen and Leunissen 1997).

Soda lakes constitute the most productive aquatic habitats in the world; supported by high ambient temperatures, light intensities and unlimited access to CO₂ (Duckworth et al. 1996). Primary productivity is due to the presence of dense blooms of alkaliphilic cyanobacteria, particularly Spirulina sp., with some contribution from alkaliphilic phototropic bacteria. The lakes are also popular habitats for different birds, in particular flamingos that feed on Spirulina. Systematic studies have shown that the microbes are alkaliphilic, many of them also being halophilic or halo tolerant (Jones et al. 1998; Duckworth et al. 2000).

Alkaliphiles, particularly those belonging to the Bacillus sp., have been extensively investigated as a source of alkali stable enzymes (Horikoshi, 1998). Within the moderately halophilic heterotrophic bacteria, the gram-positive micro organisms have been studied in less detail than the gram-negative group, represented by the genus Halomonas (Duckworth et al. 2000). Furthermore, non-motile species of the gram-positive organisms are less common, N. halobia being one representative.

The genus Nesterenkonia was first proposed on the basis of phylogenetic and chemotaxonomic differences of the strain originally described as Micrococcus halobius from the type strain of the genus Micrococcus, M. luteus (Koch et al., 1994; Stackenbradt et al., 1995). The strain, renamed as N. halobia, was first isolated from unrefined solar salt from Noda, Japan, and was recognised as a moderately halophilic gram-positive non-motile coccus (Onishi & Kamekura, 1972). It was found to produce an extra cellular amylase that was dependent on divalent cations and a high concentration of NaCl or KCl for activity and stability. The description of N. halobia was based for a long time on this single strain, which had not been characterized in detail (Ventosa et al., 1998). After a period of 25 years, detailed characterization of six cocci isolated from ponds and salterns located in Huelva, Spain, revealed phylogenetic similarity to N. halobia (Mota et al., 1997). The DNA-DNA hybridisation showed a high degree of homology (72-100%) among the six isolates and the type strain ATCC 21727. Recently, a N. halobia strain was also isolated among the starch hydrolysing alkaliphiles from Ethiopian soda lake samples (Martins et al., 2001).

A number of soda lakes are found in the Great Rift Valley running through East Africa, an arid tropical region. Soda lakes represent the most stable alkaline environments on earth with ambient pH values around 10 or higher. The lakes are characterized by high content of carbonate, low concentration of Mg²⁺ and Ca²⁺ ions (which precipitate as carbonates), and salt concentration ranging between 5-30% (w/v) (Jones et al., 1994). Investigations on the prokaryotic population of the soda lakes in the Kenyan-Tanzanian region of the Rift Valley have revealed considerable phylogenetic diversity (Duckworth et al., 1996; 2000; Grant et al., 1990; Jones et al., 1998). All the microorganisms are alkaliphilic in nature and many of them would have biotechnological potential as sources of alkali-stable enzymes (Kumar & Takagi, 1999). The soda lakes located in Ethiopia are relatively less researched as suggested by a limited number of reports in the literature (Martins et al., 2001).

In some countries, feather is used as animal feed supplement in the form of feather meal. Current commercial production of feather meal involves treatment of the feather at elevated temperature and high pressure. This process, in addition to being energy intensive, results in the loss of some essential amino acids. Moreover, the feather meal thus produced has been found to be poorly digestible by animals. Development of enzymatic and/or microbiological methods for the hydrolysis of feather to soluble peptides and amino acids is extremely attractive, as it offers a cheap and mild process for the production of valuable products. Feather may also find important application in the fermentation industry for the production of commercial enzymes.

In the past there have been some reports on the isolation of microorganisms capable of degrading keratin. Some of these micro organisms are pathogenic, such as dermatophytes, making them unsuitable for large-scale applications. To date, a limited number of studies have been reported on the isolation of feather degrading micro organisms with potential application for feather hydrolysis or for other applications. Herein it is reported on the purification and characteristics of alkaline proteases produced by two alkaliphilic bacterial strains that grow on feather as the sole source of carbon and nitrogen.

Below the characteristics of a novel moderately halophilic, alkaliphilic Nesterenkonia sp. nov, isolated from the shore of Lake Abjata, an alkaline soda lake in Ethiopia will be described as part of the present invention. In the laboratory, the isolate was able to grow on bird feather as the only source of carbon and nitrogen, and produced an extracellular calcium-independent alkaline protease.

### SUMMARY OF THE PRESENT INVENTION

AL protease, as a novel alkaline protease is called herein, is a thermostable alkaline, non-Ca²⁺-dependent protease, which has been isolated and purified from an alkaliphilic bacterium, characterized and crystallized.

In particular the invention relates to an isolated and purified alkaline protease from Nesterenkonia sp. nov. strain, having the following characteristics:

molecular weight of 23 kilodalton,

melting temperature of about 74° C. at pH 7-10,

calcium independent for activity and stability,

maximal protease activity at pH 10, and

being a serine protease.

Mud/soil sample was collected from the shore of Lake Abjata (7°60′N; 38°62′E), an alkaline soda lake in the Ethiopian Rift Valley area, as given above. About 1 g of this sample was suspended in 5 ml sterile distilled water and vigorously mixed. Aliquots of the clear suspension were streaked on to casein-yeast extract- peptone (CYP) agar plates which consist of (g/l): casein 10; peptone 5; yeast extract 1; K₂HPO₄ 1; MgSO₄·7H₂O 0.2; CaCl₂ 0.1; Na₂CO₃ 10; and agar 15 g. Na₂CO₃ was sterilised separately and added to the rest of the medium after cooling. After 48 h incubation at 37° C. protease production was observed as a clearing zone around the colony in protease positive isolates. Individual colonies were transferred to fresh agar plates, purified through repeated streaking and stored on agar slants. All Isolates, which formed a clearing zone on CYP medium, were grown in liquid medium prepared as above but in which 10 g/l chicken feathers substituted casein. The cultures were incubated at 37° C. with rotary shaking and solublization of the feather was observed visually. The level of protease production was checked from the culture supernatant obtained after centrifugation.

### DETAILED DESCRIPTION OF THE PRESENT INVENTION

The present invention relates to an isolated, novel Nesterenkonia sp. strain, herein denoted AL. Deposited at Deutsche Sammlung von Mikroorganismen und Zellkulturen under deposition number DSM 15380 on the 20th day of December, 2002 under the Budapest Treaty.

Further, the invention relates to an alkaline protease having the amino acid sequence,

N-terminal

SEQ. ID. NO. 1

QNPADSPHIGKVFFSTNQGDFVCSANIVASANQSTVATAGHCLHDGNGGQ
FARNFVFAPAYDYGESEHGVWAAEELVTSAEWANRGDFEHDYAFAVLETK
GGTTVQQQVGTASPIAFNQPRGQYYSAYGYPAAAPFNGQELHSCHGTATN
DPMGSSTQGIPCNMTGGSSGGPWFLGQGTGGAQNSVNSYGYTFLPDVMFG
PYFGSGAQQNYNYAST

and nucleotide sequence according to

SEQ. ID. NO. 2
CAG AAT CCG GCG GAC TCC CCG CAC ATA GGC AAG GTC TTC TTC TCC ACC AAC CAG
GGC GAC TTC GTC TGC TCC GCC AAC ATC GTG GCC TCG GCG AAC CAG TCC ACG GTG
GCC ACC GCG GGG CAC TGC CTG CAC GAC GGA AAC GGC GGC CAG TTC GCA CGC AAC
TTC GTC TTC GCC CCT GCC TAC GAC TAC GGC GAG TCC GAG CAC GGC GTG TGG GCC
GCA GAA GAG CTG GTG ACC TCC GCC GAG TGG GCG AAC CGC GGC GAC TTC GAG CAT
GAC TAC GCC TTC GCG GTC CTC GAG ACC AAG GGC GGC ACC ACC GTG CAG CAG CAG
GTG GGG ACG GCG TCG CCG ATC GCC TTC AAC CAG CCG CGC GGC CAG TAC TAC AGC
GCC TAC GGC TAC CCG GCC GCC GCG CCC TTC AAC GGC CAG GAG CTC CAC AGC TGC
CAC GGC ACC GCC ACG AAC GAC CCG ATG GGC AGC AGC ACT CAG GGC ATC CCG TGC
AAC ATG ACC GGC GGC TCC TCC GGC GGC CCC TGG TTc CTC GGT CAG GGG ACC GGC
GGT GCC CAG AAC TCT GTG AAC TCC TAC GGG TAC ACC TTC CTG CCG GAC GTG ATG
TTC GGG CCG TAC TTC GGC TCC GGG GCA CAG CAG AAC TAC aAC TAC GCc TCC ACA

and any sequence showing a 60% homology therewith, or preferably a 70% homology, more preferably 80% homology, and most preferably 90% homology therewith. Derivatives, as well, are contemplated, whereby such derivatives comprise the major alkaline protease activity, although the full nucleotide sequence is not present or has been completed with some other functional or non-functional derivate specie. Thus the full nucleotide sequence may have been deleted of one or more nucleotides coding for one or more amino acids, such as one to seven amino acids, whereby the basic alkaline protease activity has been retained in the molecule retained. Further, the invention relates to the application of the alkaline protease in different industrial applications, including biological laundry and dishwashing agents, leather treatment compositions and silver recovery compositions.

The invention will now be described with reference to a number of Experiments providing details of the novel alkaline protease.

Bacterial strains and culture conditions.

Strain AL was isolated from a feather sample collected at the shore of Lake Abjata, Ethiopia; 7°60′N; 38°62′E. The isolate was grown at 37° C. with shaking at 200 rpm, in complex YP medium, pH 10, containing (g l⁻¹): yeast extract (Difco, Becton Dickinson France S.A., La Pont de Claix, France), 1.0; peptone (Difco), 3.0; K₂HPO₄, 1.0; MgSO₄.7H₂O, 0.2; CaCl₂, 0.1; NaCl, 58.44 (1M); and Na₂CO₃, 10.0. Na₂Co₃ was sterilised separately and added to the rest of the medium after cooling.

Nesterenkonia halobia (DSMZ ₂₀₅₄₁T) was also grown in the medium with the same composition at 30° C. (Mota et al., 1997).

### Experiment 1

Materials and Methods 1

Light and Electron Microscopy.

Cell morphology was examined under a Nixon optiphot-2 (Nikon GmbH; Düsseldorf, Germany) phase contrast microscope at ×1000 magnification. Bacterial size was determined in living cell preparations from cultures grown on complex YP medium for 12-18 h. Gram staining was performed using a Difco Gram stain set (Difco, Detroit, Mich. USA). Scanning electron observation was conducted using a JSM-5600 LV scanning electron microscope (JEOL, USA Inc.) at ×10 000-15 000 magnifications. Cells were extracted from 12-18 h cultures grown in complex YP medium and washed twice with water and increasing concentrations of ethanol, from 40 to 100% (v/v), followed by washing with increasing concentrations of isopropyl alcohol, from 20 to 100% (v/v). Cells were then mounted on 12 mm cover slips and dried in a vacuum dessicator for 12 h and then were gold-palladium (80/20) coated.

Phenotypic Characterization.

Catalase and oxidase activities were determined as described by Smibert & Krieg (1994). Gas production in the medium supplemented with different sugars was examined using Durham tubes. Sugar fermentation pattern was determined using the API 50 CHB system (bioMerieux S. A., Marcy-I 'Etoile, France). Other biochemical characteristics were screened by API 20E system (bioMerieux), according to Logan & Berkeley (1984).

Hydrolysis of xylan and dextran was studied as described by Smibert & Krieg (1994). Starch and casein hydrolysis was tested in solid YP medium containing 1% (w/v) soluble starch and 1.5% (w/v) casein, respectively. The optimum temperature for growth was determined by incubation at 20, 30, 37, 40 and 45° C. in liquid YP medium in a pH range of 5-13. Halo tolerance was tested in the YP medium containing 0-7 M NaCl at 37° C. Sensitivity to antibiotics and tolerance to lysozyme were tested according to Smibert & Krieg (1994).

DNA Extraction and Purification.

Genomic DNA extraction was performed as described by Sambrook et al. (1989). The purity was assessed from the A₂₆₀/A₂₈₀ and A₂₆₀/A₂₃₀ extinction ratios (Johnson, 1994).

16S rDNA Sequencing.

Universal primers corresponding to position 8-27 (AGAGTTTGATCCTGGCTCAG) and 1492-1509 (GGTTACCTTGTTACGACTT), respectively, in the 165 rDNA sequence of E. coli were used to amplify 16S rDNA by PCR (Weisburg et al., 1991). The products were purified using a QIAquick PCR purification kit (QIAGEN GmbH, Hilden, Germany) and resuspended in 40 μl of sterile water. DNA sequencing of both the strands was performed by the dideoxy chain termination method with an ABI prism 3100 DNA Analyzer, using the ABI Prism BigDye Terminator Cycle Sequencing Ready Reactions kit v2.0 (PE Biosystems, Foster City, Calif., USA) according to the manufacturer's protocol.

Sequences belonging to the same genus or closely related species available through the public databases were aligned and a similarity matrix was calculated using Similarity Matrix version 1.1 software (Maidak et al., 2000). Only unambiguously aligned positions from all sequences were used to calculate the matrix, and gaps were not included in the match/mismatch count.

Peptidoglycan Type.

Analysis of the cell wall peptidoglycan type was performed according to Schleifer & Kandler (1972).

DNA Base Composition and DNA-DNA Hybridisation.

In order to calculate G+C content, DNA was isolated by chromatography on hydroxyapatite by the procedure of Cashion et al. (1977). G+C content was calculated from the ratio of deoxyguanosine (dG) and thymidine (dT) according to the method of Mesbah et al. (1989).

DNA-DNA hybridisation was carried out as described by DeLey et al. (1970) with the modifications described by Huss et al. (1983) and Escara & Hutton (1980) using a Gilford System model 2600 spectrophotometer equipped with a Gilford model 2527-R thermoprogrammer and plotter (Gilford Instrument Laboratories, Oberlin, Ohio, USA).

Renaturation rates were computed with the TRANSFER.BAS programme described by Janke (1992).

Table 1 provides a comparison of the morphological and phylogenetic characteristics of AL isolate with that of N. halobia. AL produced cream-beige, circular, convex, smooth, entire opaque colonies, 3-4 mm in diameter after 48 h of incubation at 37° C. Microscopic examination showed the cells to be non-spore forming, non-encapsulated and non-motile coryneform rods of around 0.7 μm width and 1.2 μm length. In unstained preparations, cells occur singly, in pairs; occasionally in tetrads and small, irregular clumps (FIG. 1). The isolate was Gram-positive as tested by Gram's stain reaction.

TABLE 1
Comparison of some phenotypic and biochemical properties of AL isolate
with that of Nesterenkonia halobia.

| Characteristic | AL | N. halobia DSMZ 20541^{T} |
|---|---|---|
| Gram staining | + | + |
| Cell shape | coryneform rods | coccus |
| Cell size | 0.7-1.2 μm | 0.8-1.5 μm |
| Colony colour | cream-beige | unpigmented |
| Optimal growth | 37° C. | 30° C. |
| temperature | | |
| Oxidase | + | + |
| Catalase | + | + |
| G + C mol % of DNA | 69.0 | 70-72 |
| Acid production from | | |
| L-Arabinose | − | − |
| D-Fructose | − | + |
| galactose | − | − |
| Glycerol | − | − |
| D-Glucose | − | + |
| Lactose | − | − |
| Maltose | − | − |
| D-Mannitol | − | − |
| Sucrose | − | − |
| D-Trehalose | − | − |
| D-Xylose | − | + |
| Citrate test | − | + |
| ONPG | + | − |
| Hydrolysis of | | |
| Gelatin | − | + |
| Tyrosine | − | + |
| Casein | + | + |
| Starch | + | + |
| Tyrosine | + | + |
| Tween 80 | − | − |
| Utilization of | | |
| L-arabinose | − | + |
| D-glucose | − | + |
| D-tagatose | − | + |
| Acetic acid | − | + |
| Antibiotic sensitivity | | |
| Ampicillin (10 μg) | − | + |
| Chloramphenicol (30 μg) | − | + |
| Erythromycin (15 μg) | − | + |
| Nalidixic acid (30 μg) | − | + |
| Streptomycin (10 μg) | − | + |
| Tetracycline (10 μg) | − | ND |
| Kanamicin (30 μg) | − | |

Growth of the isolate in the liquid medium was uniform, the broth was turbid, and no pellicles were produced. The isolate was strictly aerobic, mesophilic, exhibiting good growth at 30-40° C. with an optimum at 37° C. It was able to grow in the pH range of 6-11, and optimal growth was obtained at pH 10 (OD₆₀₀₀ₙₘ=6.8). Varying the NaCl concentration in the medium showed good growth at 0-2 M salt, maximal growth being observed at 1 M NaCl after 24 h of cultivation at 37° C. Moderate growth was observed in the presence of 3 M NaCl after 48 h incubation.

The strain was both oxidase- and catalase-positive. The addition of glucose, fructose or sucrose had no effect on cell growth, and also did not reveal the production of acid or gas. In contrast to N. halobia, the isolate exhibited sensitivity to all the antibiotics tested.

Analysis of the cell wall showed the presence of murein of the type L-Lys-Gly-L-Glu, variation A4α as in N. halobia (Stackebrandt et al., 1995). Meso-Diaminopimelic acid was not detected in the cell hydrolysate.

An almost complete 16S rDNA sequence was determined for AL strain, and compared with other available sequences at the GenBank and RDBP database. A similarity matrix was calculated and phylogenetic tree was constructed with the DNAMAN 4.03 Program by using the neighbour-joining method and the Jukes-Cantor distance correction method (Saitou & Nei, 1987) (FIG. 2). This analysis placed the new isolate in the Micrococcaceae family of bacteria, the closest related micro organism being Nesterenkonia halobia with 97% sequence identity.

As shown in Table 1, the G+C content of AL DNA was 69.0 mol %, implying certain affiliation to the high G+C content Gram-positive bacteria. DNA-DNA hybridisation analysis further revealed 48.5% similarity to N. halobia type strain (DSMZ ₂₀₅₄₁^{T}), hence supporting separate specie rank for the AL isolate.

Phylogenetic analysis of the soda lake isolate AL thus revealed a relatively close relationship with N. halobia. Rapid growth rates and ability to grow at high pH could decrease production costs in industrial processes by reducing contamination. The unique calcium-independent alkaline protease (see below) produced by AL protease is an interesting candidate for industrial applications.

The phylogenetic tree constructed from the similarity matrix showed AL and N. halobia to constitute a cluster separated from other species of the genera Micrococcus, Arthrobacter and Kocuria, in agreement with the earlier studies of Stackebrandt et al. (1995) and Mota et al. (1997). The A4α peptidoglycan type also supports the affiliation to Nesterenkonia genus. The AL isolate differed from N. halobia in a number of morphological and phylogenetic features (Table 1). Differences in 16S rDNA sequence identity of 3% and in the G+C content justify the proposal of a Nesterenkonia sp. nov. The hybridisation of AL against N. halobia gave reassociation value of 48.5%, which is significantly lower than the recommended threshold value of at least 70% accepted as the definition of a genospecie (Wayne et al., 1987).

On the basis of the obtained data, it is concluded that strain AL is a novel alkaliphilic and moderately halophilic Nesterenkonia specie, for which no name has been proposed so far.

The enzyme has a molecular mass of 22876±5 Da and p1 of 4.2. The optimum temperature was shown to be at 70° C. in the pH-range from 7.5 to 11.5. One of the most important characteristics of the AL protease is its calcium-independency.

### Experiment 2

Material and Methods 2

Purification of AL Protease

The AL organism was grown in an alkaline medium (pH 10) containing chicken feather as carbon and nitrogen source for 48 h at 37° C. (see below). The alkaline protease was purified from the culture supernatant according to the reported procedure comprising ammonium sulphate precipitation, ion exchange chromatography and gel filtration. Unless otherwise stated, the pure enzyme obtained (specific activity of 2200 U/mg with casein as substrate) was used for experiments with different substrates.

Hydrolysis of Oxidized Insulin B-Chain

To 0.38 mg of the oxidized insulin B-chain (Sigma) dissolved in 1 ml of 10 mM Tris-HCl buffer, pH 9 was added 10 μl (5.5 units) of the AL enzyme. The mixture was incubated at 50° C. for 15 min and 12 h, respectively, after which one drop (approx 40 μl) of 0.1% (v/v) TFA was added to inactivate the enzyme.

Identification of the cleavage products was performed on a matrix assisted laser desorption ionisation reflectron-type time-of-flight (MALDI-TOF) mass spectrometer (REFLEX, Bruker-Franzen Analytik; Bremen, Germany).

Assay with Synthetic Peptide Substrates

The AL protease activity toward a variety of commercially available synthetic peptide substrates was assayed. The incubation volume was always 1 ml in which 10 μl enzyme solution; 0.76 mg protein/ml was used unless otherwise stated. Initial reaction rates were fitted to the Michaelis-Menten equation, and kinetic parameters, k_{cat} and Kₘ were determined.

Fluorometric assay for Suc-L-Y-AMC, Suc-A-A-F-AMC, Suc-I-I-W-AMC, Suc-A-A-K-AMC, NA-Bz-R-AMC, Suc-L-L-V-Y-AMC, Suc-F-G-L-β-NNap, Suc-L-β-NNap (Sigma) was performed in 50 mM glycine-NaOH buffer, pH 10 at 25° C. The substrate concentration range was from 0.05 to 10 mM except for Suc-L-L-V-Y-AMC (0.1-25 μM). Fluorescence was monitored with a SPEX Fluorolog spectrometer, in a 1×1 cm quartz cuvette placed in a thermostat cuvette holder, using excitation wavelength of 345 nm, emission wavelength 442 nm slit width 5 nm for AMC substrate and excitation wavelength of 340 nm, emission wavelength of 410 and slit width 5 nm for β-NNap substrates. A standard solution of AMC and β-naphthylamine was used to convert the fluorescence values to product concentrations.

Activity with p-nitroanilide substrates was tested at 25° C. in 50 mM Tris-HCl buffer, pH 9. Following substrates were used: Bz-Tyr-pNA (Sigma), BAPNA, Suc-Phe-pNA, Suc-A-A-P-F-pNA, Suc-A-A-P-L-pNA, Suc-G-G-G-pNA, Suc-A-A-A-pNA, Suc-A-A-V-pNA (Bachem Feinchemikalien AG (Bubendorf, Switzerland), and S-benzyl-L-cysteine-4-nitroanilide (Fluka Chemie AG, Buchs, Switzerland). The release of p-nitroanilide (pNA) was measured at 410 nm. Extinction coefficient (E) of pNA was taken to be 8480 M⁻¹ cm⁻¹. Initial reaction rates were determined over a concentration range of 0.05-10 mM for most substrates, excluding Suc-A-A-P-L-pNA (0.05-20 mM), and S-benzyl-L-Cysteine-4-nitroanilide (0.05-80 mM).

Hydrolytic activity of the enzyme on the ester substrates, BAEE, BTEE, Bz-F-NE, p-nitrophenyl acetate, p-nitrophenyl butyrate and Suc-F-L-F-SBzl (Sigma) was examined spectrophotometrically at 410 nm (ε₄₁₀=900 M⁻¹cm⁻¹) in 50 mM Tris-HCl buffer, pH 9 at 25° C. and 50° C. respectively. The substrates concentration range between 0.05 to 10 mM. The Initial hydrolysis of BAEE, Suc-F-L-F-SBzl was followed at 253 nm (ε₂₅₃=1150 M⁻¹cm⁻¹) and BTEE at 259 nm (ε₂₅₉=964 M⁻¹cm⁻¹), at which their extinction coefficients were taken in calculation.

Due to the poor aqueous solubility of some of the substrates, their stock solutions were prepared in organic solvents, and further dilution was done with the used buffer. Suc-F-G-L-β-NNap, Suc-L-L-V-Y-AMC, Suc-L-Y-AMC, Suc-A-A-F-AMC, Suc-I-I-W-AMC, Suc-A-A-K-AMC, NA-Bz-R-AMC and Bz-Tyr-pNA, Suc-F-L-F-SBzl and Bz-F-βNE were dissolved in dimethylsulfoxide (DMSO), Suc-Phe-pNA, BTEE and BAPNA in ethanol, Suc-A-A-A-pNA in N,N-dimethylformamide (DMF), and Bz-Cys-NA in acetonitrile.

Enzyme Assay with Casein or haemoglobin as Substrate

The AL protease activity with 0.1-4% (w/v) casein and haemoglobin (Sigma), respectively, as protein substrates, was determined using a modified method of Ferrero et al. (1996). The reaction mixture in a total volume of 1 ml containing the substrate and an appropriate amount of the enzyme in 50 mM glycine-NaOH buffer, pH 10 was incubated at SOOC for 20 min, after which the reaction was terminated by addition of 0.5 ml trichloroacetic acid with subsequent centrifugation. To 0.5 ml supernatant was added 2.5 ml sodium carbonate followed by 0.5 ml of 1 M Folin-Ciocalteau Phenol reagent (Sigma). The reaction mixture was allowed to stand for 30 min-at room temperature before absorbance was measured at 660 nm. One unit of enzyme activity is defined as amount of the enzyme, which released 1 μg tyrosine per minute under the assay conditions.

Determination of Keratinolytic Activity

The hydrolysis of keratin azure (Sigma) by the AL protease was determined according to the modified method of Santos et al. (1996). The reaction mixture (1.5 ml) comprising 0.5 ml of the enzyme solution and 10 mg keratin-azure in 10 mM glycine-NaOH buffer, pH 10 was incubated at 50° C. with constant shaking over 24 h period. The substrate was removed by centrifugation and liberation of the azo dye was measured at 595 nm. The reaction under similar conditions using heat-treated enzyme (by incubation in a boiling water bath for 15 min) was also run as a control. One unit (U) of keratinolytic activity was defined as an increase in the A₅₉₅ₙₘ of 0.01 after 2 h.

The enzyme used for determination of keratin-azure hydrolysis obtained after one-step purification having a specific activity of 96 U/mg.

Measurement of Elastolytic Activity

Elastolytic activity of AL protease was estimated by the method of (Tsai et al. 1983) with slight modification. The reaction mixture in a total volume of 1.5 ml comprised of 20 mg elastin-orcein and an appropriate amount of the enzyme in 100 mM Tris-HCl buffer pH 8.8, was incubated at 37° C. for 1 h with constant shaking. The substrate was removed by centrifugation and increase in optical density was measured at 590 nm. A control reaction was performed using the denatured enzyme as described above. The concentration of soluble products liberated was calculated from a standard curve using elastase. One unit was defined as the amount of enzyme, which solubilizes 100 μg of elastin under standard reaction conditions.

Measurement of Protein Concentration

Total protein concentration was determined by the bicinchoninic acid (Sigma) method using bovine serum albumin (Sigma) as the standard protein.

Specificity of the AL Protease on Oxidized Insulin B-Chain

Cleavage of oxidized insulin B-chain by the AL protease at 50° C. was investigated using an enzyme-substrate molar ratio of 1:100. The reaction products were analyzed by MALDI-TOF mass spectrometry and identified from the mass of corresponding peptide pairs.

To identify the primary cleavage sites, the reaction was stopped after 15 min. The oxidized insulin was cleaved at Phe²⁵-Tyr²⁶, liberating peptide fragment 293Z.2 Da (FIG. 3a); this fragment was further cleaved between Leu¹⁵ and Tyr¹⁶, producing two smaller fragments of 1238 and 1714 Da (FIG. 4). The enzyme further acted on insulin B-chain producing peptide fragments of 1350.9 Da (FIG. 3a) suggesting a cleavage on carbonyl bond of Gln⁴, Tyr¹⁶ and Tyr²⁶ (FIG. 4). Since the peptide fragments derived Leu¹⁵-Phe²⁵ and Tyr¹⁶-Tyr²⁶ possess the same molecular mass (1238 Da) and if we take the localization of the fragments Phe¹-Phe²⁵ and Gln⁴-Tyr¹⁶ into consideration, it seems reasonable to suggest that the peptide 1238 Da in MALDI spectra correspond to both fragment Tyr¹⁶-Phe²⁵ and Leu¹⁷-Tyr26, suggesting the cleavage site on carbonyl bond of Tyr²⁶ (FIG. 4).

Secondary hydrolysis was studied after 12 h of incubation. Besides the previously obtained peptides the fragments with molecular masses 1791.1, 1910.1 and 1167.2 Da (FIG. 4) were librated. Results indicate that the oxidized insulin was first cleaved between Leu¹⁵ and Tyr¹⁶ producing two fragments with molecular masses of 1714 and 1791.1 Da respectively. These two were further cleaved at Gln⁴and Phe²⁵ generating fragments of 1167.2 and 1238 Da respectively. The peptide fragment with molecular mass of 1910.1 Da indicates the additional cleavage sites at Ser⁹-His¹⁰ bond (FIG. 4). Since MALDI does not detect fragment with less than 500 Da molecular masses the small fragments were not appearing in MALDI spectra.

The results indicated that the enzyme initially cleaves the Leu¹⁵-Ty¹⁶, which is a common preferable cleavage site among bacterial serine alkaline proteases (Tsuchiya et al. 1993). The cleavage site at Gln⁴-H is⁵ and Ser⁹-His¹⁰ Tyr¹⁶-Leu¹⁷, Tyr²⁶ -Th²⁷ in the oxidized insulin B-chain by AL protease were identical with those on the work with subtilisin Carlsberg and Novo; the enzyme cleaved Phe²⁵-Tyr²⁶ as subtilisin BPN′.

Specificity of AL Protease with Synthetic Substrates

Simultaneously, substrate specificity of the AL protease was evaluated with synthetic peptide substrates, pNA, AMC, NNap, and esters. The fluorogenic peptide substrates (AMC) have earlier been used for determination of protease specificity. Suc-A-A-P-F-pNA has been used as a specific substrate for subtilisin and Suc-A-A-P-L-pNA as elastase specific substrate.

The kinetic parameters for hydrolysis of synthetic substrates by AL protease are summarized in Table 2. The enzyme showed remarkable hydrolyzing activity towards Suc-L-L-V-Y-AMC, Suc-A-A-P-F-pNA and Suc-A-A-P-L-pNA substrates, but was unable to hydrolyze Suc-A-A-A-pNA, Suc-A-A-V-pNA, Suc-G-G-G-pNA and NNap substrates. The k_{cat}/Kₘ value for Suc-L-L-V-Y-AMC was approximately 2.5-5 fold higher than that for Suc-A-A-P-F-pNA and Suc-A-A-P-L-pNA, which could partly be a result of decrease in Kₘ due to the higher binding affinity for Suc-L-L-V-Y-AMC.

TABLE 2

| Substrate | Km (mM) | kcat (s-1) | kcat/Km (mM-1 · s-1) |
|---|---|---|---|
| Suc-L-L-V-Y-AMC^{a} | 0.015 | 51.68 | 3445.30 |
| Suc-A-A-P-F-pNA^{b} | 2.97 | 4391.46 | 1478.61 |
| Suc-A-A-P-L-pNA^{b} | 9.3 | 6646.19 | 714.64 |
| Benzyl-Cys-4-pNA^{b} | n.d | n.d | 34.50 |
| BTEE^{b} | n.d. | n.d. | 62.00 |
| BAEE^{b} | n.d. | n.d. | 168 |
| p-Nitrophenyl butyrate^{b} | 1.05 | 63.74 | 60.76 |

The high catalytic activity of AL protease toward Suc-L-L-V-Y-AMC, Suc-A-A-P-F-pNA and Suc-A-A-P-L-pNA substrates are consistent with most of the subtilisins that show specificity for substrates containing Pro-Phe or Val-Tyr at the P2-P1 sites. The Kₘ values determined for Suc-A-A-P-F-pNA and Suc-A-A-P-L-pNA were large in comparison to subtilisin Carlsberg and BPN′, however kcat values and thus the k_{cat}/Kₘ values were higher. This suggested that the cleavage of the enzyme-substrate complex in case of AL protease was much faster although the substrate binding affinity was low.

The activity of AL protease toward a variety of smaller pNA and AMC substrates was examined. Activity was detected with only a few substrates; the examples are Suc-A-A-F-AMC, Suc-F-L-F-SBzl, and Suc-L-Y-AMC. However, the enzyme was only able of hydrolysing those substrates at a very low rate when high substrate concentration (10 mM) was used. The specific activity of AL protease toward those substrates was between 0.002 to 0.008 U/mg (data not shown).

The AL protease was not very effective against a number of trypsin and chymotrypsin-type substrates (e.g. BTEE, BAEE, BAPNA and Suc-Phe-pNA). The enzyme did not show detectable activity with BAPNA and Suc-Phe-pNA, the activity with BTEE and BAEE was observed only after adding higher amount of the enzyme.

The individual Kₘ and kct for BAEE, BTEE and Benzyl-Cys-4-NA could not be determined due to weak binding of the substrates. In these cases the k_{cat}/Kₘ was calculated from velocity measurements at low substrate concentration [S]<<Kₘ, where v=(k_{cat}/Kₘ)[E]₀ [S].

The cleavage of p-nitrophenyl butyrate was obtained only after 1 h incubation at 50° C. while the enzyme was unable to cleave p-nitrophenyl acetate at all. A similar observation has been reported earlier for Subtilisin Carlsberg.

The higher amount of the enzyme (up to 15 fold), longer time of incubation and different temperature was used for the substrates for which activity was not detected. Poor activity of AL protease toward smaller substrates could be attributed to a low binding affinity of the enzyme to these substrates and/or that the enzyme prefers longer-chained substrate to express the best activity. Moreover, catalytic efficiency analysis of AL protease toward several differently sized substrates has shown that when the peptide portions of substrates become shorter, the Kₘ values increase.

Degradation of Various Protein Substrates by the AL Protease

Hydrolysing effect of AL protease on haemoglobin and casein was determined using different concentration of the substrates; casein and haemoglobin were completely hydrolysed within 20 min (FIG. 6). The reaction followed Michaelis-Menten kinetics.

The degradation of insoluble protein substrates by the AL protease was determined by measuring hydrolysis of keratin-azure and elastin-orcein. The main hydrolysis of keratin-azure was observed within 2-12 hours of incubation (FIG. 5). Further incubation and/or increase in amount of the enzyme did not result in additional degradation of the protein.

The enzyme was able to hydrolyse elastin-orcein to a detectable level. The relationship between enzyme concentration and dissolved elastin-orcein was quite linear when different amount of enzyme was used (data not shown).

The obtained results on the cleavage patterns of the oxidized insulin B-chain and synthetic substrates were in good agreement. Combining results exhibit the specificity of the enzyme for hydrophobic and aromatic amino residues such as Tyr, Phe and Leu. Four of the six cleavage sites in insulin B-chain were aromatic and/or hydrophobic residues which can point out that the AL protease prefers bonds with aromatic and hydrophobic amino acids in the P1 site (FIG. 4). However, the other hydrophobic and aromatic residues present in the oxidized insulin B-chain that were not cleaved by AL protease may be attributed to inhibition at the other sub-sites such as P2 and/or the length of the peptide fragment. These were supported by the increased specificity of the enzyme for large synthetic substrates containing aromatic or hydrophobic amino acids (e.g. Phe or Tyr) at P1 site and neutral residues (e.g. Val, Pro) at P2 site. The catalytic efficiency of AL protease was enhanced by an aromatic residue (Phe) at P1 position compared to aliphatic amino acids (Leu) at the same site (Table 2). Besides the substrate composition, the substrate size appeared to be important for the determination of the enzyme specificity. The cleavage of larger substrates by the AL was significant while the enzyme could cleave the shorter substrate at low rate.

The alkaline protease-producing strain AL was shown to effectively hydrolyse and grow on feather at 37° C. It is possible that initial selection of the strain for the ability to grow on feathers may have yielded the enzyme with enhanced specificity for aromatic and hydrophobic substrates. The specificity of the enzyme for those amino acids may play an important role in the degradation of feather keratin since 50% of the residues found in keratin molecule are hydrophobic or aromatic (Gregg et al. 1984). The characteristic of keratin is its high resistance to proteolytic digestion. The resistance is due to the super coiled helical structure of polypeptide chain, high degree of cross-linking by cystine disulfide bond, hydrophobic interaction, and hydrogen bonding. One of the possible mechanisms in breaking down keratin is the reduction of disulfide bonds. Enhanced keratin degradation after addition of disulfide-reducing agent such as dithiothreitol and thioglycolate has been reported for serine proteases. However, such a mechanism is not likely in the case of AL protease since it degrades keratin without a reducing agent or coenzyme. Biodegradation of feather by this bacterium and produced enzyme can play an important role in biotechnological application like enzymatic improvement of feather meal, production of amino acids and peptide from high molecular weight substrates and in the leather industry.

The AL protease not only could hydrolyze keratin but was able to hydrolyze a variety of soluble and insoluble protein substrates, as well. The soluble substrates casein and hemoglobin were readily degradable, while the insoluble substrate elastin was less so.

The AL protease is thus a thermostable alkaline protease with ability of cleaving oxidized insulin B-chain, some peptide substrates and soluble and insoluble protein substrates.

The enzyme is highly active against large substrates containing aromatic or hydrophobic amino acids at P1 site and neutral residues at P2 site. The catalytic efficiency (k_{cat}/Kₘ) significantly increases when the hydrophobicity of amino acid residue positioned at P1 site increases. In addition it seems that the specificity of the enzyme to aromatic and hydrophobic amino acid residues play an important role in the enzyme capability of hydrolysing insoluble proteins such as keratin.

The alkaline protease AL above was subjected to a comparative study with another alkaline protease from a Bacillus strain. The two alkaline proteases producing alkaliphilic bacterial strains, designated as AL and AL-89, respectively, were isolated from a naturally occurring alkaline habitat. The two strains were identified as Nesterenkonia sp. AL and Bacillus pseudofirmus, respectively. Both strains grew and produced alkaline protease using feather as the sole source of carbon and nitrogen. B. pseudofirmus AL-89 grew well and produced significantly higher level of protease activity using feather medium. Addition of 0.5% glucose to the feather medium increased protease production by B. pseudofirmus AL-89 and suppressed enzyme production by Nesterenkonia sp. AL. The enzymes from both organisms were purified to electrophoretic homogeneity following ammonium sulphate precipitation, ion exchange, hydrophobic interaction, and gel filtration chromatography. The molecular weight, determined using SDS-PAGE, was 23 kD for AL protease and 24 kD for AL-89 protease. The AL protease was active in a broad pH range displaying optimal activity at pH 10 and over 90% of its maximum activity between pH 7.5 to 11.5. The enzyme is unique in that unlike all other microbial serine proteases known so far, it did not require Ca²⁺ ions for activity and thermal stability. Its optimum temperature for activity was at 70° C. and showed very good stability after 1 h incubation at 65° C. irrespective of the presence or absence of Ca²⁺.

These characteristics make protease AL an ideal candidate as additive in detergents that contain chelating agents to overcome water hardness. The AL-89 protease on the other hand, required Ca²⁺ for activity and stability at temperatures above 50° C. Its optimum activity was at 60° C. and 70° C. in the absence and presence of Ca²⁺, respectively. It displayed a pH optimum of 11 and retained about 70% or more of its original activity between pH 6.5 and 11.

Feather is composed of over 90% protein, the main component being keratin, a fibrous and insoluble protein highly cross-linked with disulphide and other bonds. In a mature chicken, feather accounts up to 5-7% of the live weight. World wide, several million tons of feather are generated annually as waste by poultry processing industries. Considering its high protein content, this waste could have a great potential as source of protein and amino acids for animal feed and for many other applications. However, because of the insoluble nature of keratin and its resistance to enzymatic digestion by animal, plant and many known microbial proteases, use of feather as a source of value added products has been very limited. Rather the large amount of feather produced and its localised accumulation around poultry processing sites create a serious disposal problem leading to environmental pollution.

### Experiment 3

Materials and Methods 3

Chicken feather was obtained from a local poultry-processing industry, Kronfagel, Sweden. The feather was collected immediately after slaughtering of the birds and repeatedly washed with distilled water until the effluent was very clear. After drying, the large feather stocks were cut by hand into smaller pieces to fit to the culture flask. DEAE-Sepharose, butyl-Sepharose, and Sephadex G-75 were obtained from Amersham Pharmacia Biotech, Uppsala, Sweden. All other chemicals and bacteriological media were from standard sources.

Enzyme Production

One hundred ml of feather medium in 500 ml baffled flasks was inoculated with 2 ml of a 48 h culture grown under similar conditions. After 48 h the culture was centrifuged to remove cells and Insoluble residues, and the culture supernatant was used as the enzyme source.

Enzyme Purification

The cell free culture supernatant was supplemented with solid ammonium sulphate to 60% saturation (for AL-89) or 70% saturation (for AL), centrifuged, and resuspended In 10 mM Tris-HCl buffer, pH 8 and dialysed against the same buffer. The concentrated enzyme preparations were applied to DEAE-Sepharose column (2.5×12 cm) and the bound enzyme was eluted using a linear gradient of NaCl (0-0.5 M for AL-89 and 0-0.7 M for AL) in 10 mM Tris-HCl buffer. Fractions containing protease activity were pooled and dialysed. To protease AL sample was added 0.5 M ammonium sulphate for application on butyl Sepharose column (1.5×9 cm), pre-equilibrated with 10 mM Tris-HCl buffer, pH 8 containing same concentration of ammonium sulphate. After washing the column to remove unbound proteins, the enzyme was eluted with a linear gradient of 0.5 to 0 M ammonium sulphate in the same buffer as above. After dialysis and concentration the pooled enzyme fractions of AL-89 protease from DEAE-Sepharose column and AL protease from butyl-Sepharose column were applied on Sephadex G-75 column (1×80 cm) for size-exclusion chromatography.

Enzyme Assay

Protease activity was determined using casein as substrate as described earlier [3.12]. The reaction mixture in a total volume of 2 ml was composed of 1% casein, 50 mM glycine-NaOH buffer, pH 10, and appropriately diluted enzyme. After 20 min incubation at 50° C. the reaction was terminated by adding equal volume of 10% trichloroacetic acid (TCA). After separation of the unreacted casein precipitate by centrifugation, 0.5 ml of clear supernatant was mixed with 2.5 ml of 0.5 M Na₂CO₃ and 0.5 ml of 1 N Folin-Ciocalteau's phenol reagent. After 30 min at 25° C., absorbance was measured at 660 nm against a reagent blank. One unit of protease activity was defined as the amount of enzyme that released 1 μg of amino acid equivalent to tyrosine per min under the standard assay conditions.

Isolation and Screening of the Organisms

AL and AL-89 (see above) were selected for further study. The two strains were characterised based on 16S rRNA sequence, fatty acid profile and biochemical characterisation at the Deutsche Sammiung von Mikroorganismen und Zelikulturen GmbH (DSMZ), Mascheroder Weg 1B, 38124 Braunschweig, Germany. Based on biochemical characterization, fatty acid analysis, and 16S rRNA sequence comparison, strain AL-89 was identified as Bacillus pseudofirmus, while strain AL exhibited closest (96.6%) 165 rRNA sequence identity to Nesterenkonia halobia.

Enzyme Production and Purification

Both strains grew and produced alkaline protease using chicken feather as the sole source of nitrogen and carbon. Complete solubilization of feather was observed after 48-60 h. Bacillus pseudofirmus AL-89 produced high level of protease activity in feather medium and solubilized feather much more efficiently than Nesterenkonia sp. AL (Table 3). Both strains produced protease activity when grown using feather or casein as nitrogen and carbon source. However, for both strains the level of protease production was higher when they were grown on feather than on casein. In fact, AL organism gave no detectable protease activity with casein in the absence of other nitrogen supplements. Increased level of protease production by Bacillus pseudofinnus AL-89 was observed on addition of glucose whereas for Nesterenkonia sp. AL protease production was suppressed in the presence of glucose (Table 3).

TABLE 3
Protease production by Nesterenkonia sp. AL and Bacillus pseudofirmus
AL-89 grown on feather and casein media in the presence of different
supplements

| Basal Medium^{a} | | Protease production (U/ml) | |
|---|---|---|---|
| plus | Supplement | AL | AL-89 |
| Feather | None | 42.1 | 238.3 |
| | Trace elements | 47.8 | 311.2 |
| | Glucose | 6.1 | 452.8 |
| | Yeast extract | 53.1 | 236.2 |
| | Peptone | 101.5 | 222.4 |
| | Yeast extract + peptone | 81.4 | 270.6 |
| Casein | None | 0 | 66.6 |
| | Peptone + glucose | 61.4 | 400.9 |
| | Yeast extract | 63.4 | 159.7 |
| | Peptone + yeast extract | 79.9 | 111.7 |

^{a}The basal medium was composed of (g/L): K₂HPO₄ 1.0; CaCl₂ 0.1; MgSO₄.7H₂O 0.2; and Na₂CO₃ 10. All the other supplements were included at a final concentration of 0.5%.

The proteases from the two strains were purified to electrophoretic homogeneity following ammonium sulphate precipitation, ion exchange chromatography, hydrophobic interaction chromatography (only for AL) and gel filtration chromatography. A summary of the purification of the two enzymes is shown in Table 4. The purified enzymes of both proteases appeared homogeneous on SDS-PAGE performed on a 12% acryl amide gel (data not shown). The molecular weight of protease AL and protease AL-89 were estimated to be 23 kD and 24 kD, respectively, which also confirmed data submitted above.

TABLE 4
Summary of purification procedures for the two
proteases AL and AL-89

| | | | | | |
|---|---|---|---|---|---|
| | Total | Total | Specific | | |
| | activity | protein | activity | Purification | Yield |
| Sample | (U) | (mg) | (U/mg) | fold | (%) |

a) Nesterenkonia sp. AL protease

| | | | | | |
|---|---|---|---|---|---|
| Culture filtrate | 325050.0 | 7890.0 | 41.2 | 1 | 100 |
| Salt | 271252.5 | 615.0 | 441.1 | 10.7 | 83.4 |
| precipitation | | | | | |
| Ion-exchange | 166878.4 | 158.1 | 1036.5 | 25.2 | 50.4 |
| chromatography | | | | | |
| Hydrophobic | 163692.4 | 105.3 | 1554.5 | 37.8 | 50.3 |
| interaction | | | | | |
| chromatography | | | | | |
| Size exclusion | 143462.0 | 79.6 | 1802.3 | 43.7 | 44.1 |
| chromatography | | | | | |

b) Bacillus pseudofirmus AL-89 protease

| | | | | | |
|---|---|---|---|---|---|
| Culture filtrate | 135180.0 | 4398.0 | 30.7 | 1.0 | 100 |
| Salt | 83513.3 | 1824.0 | 45.8 | 1.5 | 62 |
| precipitation | | | | | |
| Ion-exchange | 32463.2 | 49.4 | 657.1 | 21.4 | 24 |
| chromatography | | | | | |
| Size exclusion | 2427.3 | 3.5 | 693.5 | 22.6 | 18 |
| chromatography | | | | | |

Effect of pH on Activity and Stability

The pH profile of the two proteases determined at 50° C. is shown in FIG. 7. Both proteases were optimally active in the alkaline region. AL protease was active in the pH range of 7.5 to 11.5 with an optimum at pH 10, and displayed about 60% of its peak activity at pH 12 (FIG. 7A). AL-89 protease exhibited optimum activity at pH 11 and low activity below pH 10 and above 11.5 (FIG. 7B).

The effect of pH on enzyme stability was studied by incubating the enzyme in different buffers of varying pH values at 50° C. for 1 h and then measuring the residual activity using standard assay procedure. AL protease was very stable in a broad pH range, maintaining over 90% of its original activity between pH 6 and 11, and more than 70% activity at pH 12 (FIG. 8A). AL-89 protease was less stable retaining about 70% or more of its original activity between pH 6.5 and 10.5, and much lower activity beyond this pH range (FIG. 8B).

Effect of Temperature on Activity and Stability

The effect of temperature on activity of the two proteases was determined by assaying enzyme activity at different temperatures in glycine-NaOH buffer, pH 10, while the thermal stability was determined by incubating the enzymes at different temperatures followed by measuring the residual activity under standard conditions. These measurements were done both in the presence and absence of 5 mM Ca²⁺. The AL protease showed a unique property in being Ca²⁺ independent with respect to its activity and stability at all the temperatures tested (FIG. 9A, 10A). Its optimum temperature for activity was at 70° C. (FIG. 9A), but when incubated at that temperature the enzyme was inactivated within 1 h (FIG. 10A). On the other hand, AL-89 protease was Ca²⁺ dependent, the optimum activity being 60° C. and 70° C. in the absence and presence of Ca²⁺, respectively (FIG. 9B). At temperature values above 50° C. Ca²⁺ was absolutely required for the thermal stability of AL-89 protease (FIG. 10B).

Effect of Protease Inhibitors

Table 5 shows the effect of different protease inhibitors on the activity of the two proteases. AL-89 protease was totally inhibited in the presence of 1 mM PMSF, a serine protease inhibitor. On the other hand, PMSF at a concentration of up to 10 mM did not inhibit AL protease activity. AL protease was also not inhibited by inhibitors of cysteine proteases, aspartate proteases and metal proteases (Table 5). The effect of another serine protease specific inhibitor, 3,4-dichlorocoumarin at a concentration of 100 μM on the protease activity was investigated. The enzyme lost 70% and 90% of its original activity after 1 h and 3 h incubation, respectively, indicating that it is a serine protease (Table 5).

TABLE 5
Effect of different inhibitors on the activity of AL protease and AL-89
protease^{a}

| | | Remaining activity (%) | |
|---|---|---|---|
| Inhibitor | Concentration (mM) | AL | AL-89 |
| None | — | 100 | 100 |
| EDTA | 10 | 99 | 98 |
| PMSF | 1 | 100 | 0 |
| PMSF | 10 | 108 | 0 |
| 3,4-DCI (after 1 h) | 0.1 | 30 | ND |
| 3,4-DCI (after 3 h) | 0.1 | 11 | ND |
| Pepstatin | 0.1 | 102 | 100 |
| IAA | 5 | 100 | 94 |
| PCMBA | 5 | 99 | 91 |
| 1,10-Phenanthroline | 10 | 113 | 75 |

^{a}The enzyme was first incubated with the inhibitor for 1 h (or up to 3 h for 3,4-dichlorocoumarin) at 30° C. Residual activity was measured following standard assay procedure.
ND: Not determined.

An interesting potential application provided is that feather as well as hair, a cheap and readily available substrate, can be used for the production of alkaline proteases at the industrial level. Currently over 30% of the total industrial enzyme market is accounted for by alkaline proteases used for detergent, leather tanning, and food applications (Bott, R. (1997); Egmond, M. R. (1997); Kalisz H. M. (1988)). In the production of industrial enzymes, up to 30-40% of the production cost is accounted for by the growth substrate. Therefore, the use of feather, a cheap and readily available substrate, could result in a substantial reduction in the cost of enzyme production. However, aspects like transportation of the feather, etc. need to be considered while considering such an application.

Because of the high carbonate/bicarbonate concentration, East African alkaline soda lakes have low calcium and magnesium concentration due to the formation of insoluble salts. As a result, extracellular enzymes from micro organisms adapted in these environments are expected to require low concentration or no Ca²⁺ ions. Different studies have shown that subtilases (the subtilisin super family of proteases) contain bound Ca²⁺ which play an important role in stabilizing the enzyme against thermal denaturation and autodegradation. For example, protease like subtilisin BNP′ of Bacillus amyloliquefaciens, protease K and aqualysin have one strong and one weak Ca²⁺ binding site. In these enzymes, removal of Ca²⁺ from the weak binding site is associated with significant reduction in thermal stability. In the process of detergent formulation, where alkaline proteases are commonly added, chelating agents are included to overcome the problem of water hardness. In the presence of such chelating agents, however, the Ca²⁺ from the weak binding site can easily be stripped off thus greatly affecting the thermal stability of the detergent enzyme under application conditions. Therefore, enzymes that do not require Ca²⁺ for stability could offer tremendous potential for detergent application. Increasing the calcium binding efficiency or deletion of the calcium binding loop through protein engineering has been tried as alternatives to improve the thermal stability of subtilisin. As demonstrated in this study another alternative, which has not been given any significant attention to date, could be to look for new enzymes from Ca²⁺-deficient or low calcium habitats, such as alkaline soda lakes of East Africa. The protease produced by Nesterenkonia sp. AL requires no Ca²⁺ ions for activity and stability.

This may suggest an evolutionary adaptation to a low Ca²⁺ environment, which could be a result of strong Ca²⁺ binding or absence of any bound Ca²⁺ on the enzyme molecule. Considering its Ca²⁺ independence for stability, its broad pH range for activity, and its other properties, protease AL is an interesting candidate for detergent application. Moreover, further understanding of the structure of this enzyme and the mechanism of its Ca²⁺-independent thermal stability may give a better insight for the rational design of new alkaline proteases through protein engineering.

Furthermore, as these enzymes act on feather suggest their ability to bind to the keratinaceous solid substrate. Keratinaceous soil is often encountered in laundry, such as shirt collars. Hence, detergent enzymes are expected to act on proteinaceous substrates attached to solid surfaces.

In conclusion, the activity and stability of AL protease in a broad pH range, its stability at high temperature in the absence of Ca²⁺, and its ability to bind to the solid feather substrate and its ability to hydrolyse keratinaceous substrate makes it extremely attractive for detergent application. This study also demonstrated the potential usefulness of feather both as a cheap fermentation substrate for the production of industrial enzymes and as a source of valuable amino acids and soluble proteins that can be used as animal feed supplements.

The protease was produced by the organism in an alkaline medium (pH 10) containing feather as the only source of carbon and nitrogen. The enzyme was seen to be optimally active at 70° C. and in the pH-range of 7.0 to 11.0. Furthermore, unlike other alkaline proteases, the enzyme activity was found to be independent of the presence of calcium ions.

A study of thermodynamic stability of the AL alkaline protease performed under different conditions, in view of its potential as a detergent additive, is given below. As a comparison Subtilisin Carlsberg, one of the first commercial alkaline proteases was used.

### Experiment 4

Materials and Methods 4

Production of AL Protease

Nesterenkonia sp. AL was grown at 37° C. for 48 h in an alkaline medium containing chicken feather as the carbon and nitrogen source (see above). The alkaline protease was purified from the culture supernatant according to the reported procedure comprising ammonium sulphate precipitation, ion exchange chromatography, and gel filtration. The pure enzyme was recovered with a yield of 57% and purification factor of 48-fold, having a specific activity of about 2200 U/mg.

Preparation of Ca²⁺-Free AL Protease

One millilitre of the purified AL protease (3 mg protein) solution was dialyzed for 24 h at 4° C. against 1 L of 10 mM glycine-NaOH buffer, pH 10.0, pretreated for 48 h with Chelex 100 (BioRad, Hercules, Calif., USA; Sg/l) placed in a dialysis bag. Thereafter the dialysis was repeated three times against the buffer containing Chelex 100 (1.5 g/l) for a total period of 36 h at 4° C. The buffer was prepared from deionised water and plastic containers were used throughout. The residual calcium content In the enzyme sample was determined using Inductively Coupled Plasma-Mass Spectrometry (ICP-SMS).

Circular Dichroism Spectroscopy

The conformational stability of the AL protease was examined by circular dichroism (CD) spectroscopy and compared with that of Subtilisin Carlsberg (bacterial protease, type VIII; Sigma Chemical Co., St. Louis, USA). The CD spectra of the proteins were obtained on a Jasco spectropolarimeter model J-720, at 25° C. in the far UV range from 195 to 250 nm at a scan rate of 10 nm/min using a sample cell path length of 0.1 cm. The ellipticity values (θ) for every nm wavelength increase were obtained in mdeg directly from the instrument and were recorded online in a computer. An average of 6 scans was calculated and the corresponding no-enzyme spectra were subtracted for each sample.

Differential Scanning Calorimetry

The influence of different parameters including pH, calcium ions, EDTA, and detergent on thermal stability of the AL protease was investigated by differential scanning calorimetry (DSC). Comparative studies were also performed with Subtilisin Carlsberg. Thermal scans of AL protease (0.3 mg protein/ml) and subtilisin (2.5 mg protein/ml in the presence of 1 mM phenyl-methanesulfonyl fluoride (PMSF); Sigma) were performed by increasing temperature from 25° C. to 95° C. at a scan rate of 1° C. per min on a VP-DSC (MicroCal, Northampton, Mass.). The thermograms were corrected by subtracting the base line, obtained from a blank scan of the no-enzyme solution, and normalized for protein concentration. The unfolding transition of the AL protease was fitted to a two-state model. The Tₘ (temperature at which heat capacity change is a maximum), the calorimetric (or true) enthalpy ΔH_{cal} (peak area), and van't Hoff (or apparent) enthalpy AHVH (calculated from the shape of thermogram) were calculated using the Origin software provided by MicroCal.

SDS-Polyacrylamide Gel Electrophoresis

Autolytic degradation of the protease was investigated by SDS-PAGE. The AL protease and Subtilisin Carlsberg were individually incubated in 10 mM Tris-HCl buffer 8 for 24 h at 50° C. Electrophoresis of the enzyme samples taken at different time intervals was performed according to the method of Laemmli, on 12% polyacrylamide gel using a Mini Protean II equipment (Bio-Rad, Richmond, Calif.).

Protease Assay

Assay of protease activity was performed according to Ferrero et al. with some modifications. One millilitre of the reaction mixture containing 1% casein in 50 mM glycine-NaOH buffer, pH 10.0 and an appropriate amount of enzyme was incubated at 50° C. for 20 min after which the reaction was terminated by addition of 0.5 ml of trichloroacetic acid and centrifuged. 2.5 ml sodium carbonate was added to 0.5 ml supernatant followed by 0.5 ml of 1 M Folin-Ciocalteau reagent. The reaction mixture was allowed to stand for 30 min at room temperature before measuring the absorbance at 660 nm. One unit of protease activity was defined as the amount of enzyme, which released 1 μg tyrosine per min under standard assay conditions.

Temperature Stability

FIG. 11 shows the circular dichroism spectrum of the AL protease and Subtilisin Carlsberg at pH 10.0 in glycine-NaOH buffer in the range of 195-250 nm. From the CD signal of the AL enzyme, its secondary structure appears to be a mixture of α-helix and β-sheet (FIG. 11a). The characteristic CD features of α-helix structure are two negative bands at ca. 222 and 208 nm while that of β sheet structure is a negative band at ca. 215 nm. The AL protease secondary structure was undisturbed after incubation for 24 h at 50° C. (FIG. 11a). However, Subtilisin Carlsberg underwent a substantial loss of structure under similar conditions (FIG. 11b). Gel electrophoresis of both the enzymes after incubation for different time periods at 50° C. revealed considerable degradation of subtilisin within initial few hours while AL protease profile was unaltered even after 24 h (not shown).

Thermal denaturation of the AL protease at increasing temperature was then studied by differential scanning calorimetry. Similar thermograms were obtained under all conditions and repeated scans of the same protein sample indicated irreversible denaturation. The enzyme concentration (0.1-0.6 mg protein/ml) had no effect on the thermal denaturation. As seen in Table 6, the melting temperature, Tₘ i.e. the midpoint in the thermally induced transition from the folded to the unfolded state, was almost constant around 74° C. in a pH range of 8.0-10.0. Beyond this pH range, only a slight decrease in Tₘ was observed reaching 69.7° C. at pH 6.0 and 72.0° C. at pH 11.0. The denaturation enthalpy, ΔH_{cal} of unfolding for the AL protease followed a similar trend, being 158 kcal/mol at pH 10.0 and decreasing to 141 kcal/mol at pH 6.0 and 142 kcal/mol at pH 11.0 (Table 6). The mean ratio of the calorimetric enthalpy, ΔH_{cal} to van't Hoff enthalpy, ΔH_{vH} was close to unity (±0.05) at any given pH.

TABLE 6
Dependence of denaturation temperature for AL protease on pH

| PH | Tₘ (° C.) | ΔH_{cal} (kcal/mol) | ΔH_{vH} (kcal/mol) | ΔH_{cal}/ΔH_{vH} |
|---|---|---|---|---|
| 6 | 69.7 ± 0.02 | 141.0 ± 0.5 | 150.0 ± 1.2 | 0.94 |
| 7 | 73.5 ± 0.02 | 149.0 ± 0.5 | 147.0 ± 1.2 | 1.02 |
| 8 | 74.3 ± 0.02 | 158.0 ± 0.5 | 171.0 ± 1.2 | 0.93 |
| 9 | 74.5 ± 0.02 | 157.5 ± 0.5 | 155.0 ± 1.3 | 1.02 |
| 10 | 74.5 ± 0.03 | 158.0 ± 0.5 | 167.0 ± 1.7 | 0.95 |
| 11 | 72.6 ± 0.02 | 141.5 ± 0.5 | 141.0 ± 1.3 | 1.01 |

The Ca²⁺ content in AL protease sample at pH 10.0 was determined to be around 0.143 mol/mol enzyme, which was reduced to 0.109 mol/mol after treatment with chelex. DSC scan of the enzyme pre-treated with chelex, and in the presence of 5 mM Ca²⁺ and 10.0 mM ethylenediamino tetraacetate (EDTA), respectively, at pH 10.0 showed the melting profile and the denaturation temperature to be unaltered (FIG. 12a). Similar results were obtained also at pH 8.0 (data not shown). The ΔH_{cal} remained constant around 158±0.5 kcal/mol in all cases.

In contrast to the AL enzyme, the thermal scans of Subtilisin Carlsberg needed to be performed in the presence of 1 mM PMSF, the serine protease inhibitor in order to prevent degradation of the enzyme during the heating process. The Tₘ of the enzyme was 67.3° C., which remained unchanged on addition of Ca²⁺ to the sample. This is because the Subtilisin already has bound Ca²⁺ and further addition of metal ions has no effect on thermal stability of enzyme. The Tₘ was, however, lowered by 10° C. in the presence of 10 mM EDTA and so was ΔH_{cal} from 203 to 152 kcal/mol (FIG. 12b). DSC of the enzyme treated to remove bound calcium did not provide any melting profile whatsoever, probably due to its autolysis.

Compatibility with Detergent Components

DSC was also used to compare the effect of 0.1-1% (w/v) sodium dodecyl sulphate (SDS) on thermodynamic stability of AL protease and subtilisin in 10 mM glycine-NaOH buffer, pH 10.0 (Table 7). Only at the detergent concentration of 1% did the stability of AL protease start to get slightly affected, with a decrease in Tₘ by 1.4° C. and in ΔH_{cal} by 4 kcal/mol. These results were in good agreement to the detergent effect on the enzyme activity. About 75% of the initial activity was retained after 90 min incubation with 1% SDS at 40° C., pH 10.0. In contrast, subtilisin exhibited high instability, Tₘ decreasing by 13° C. and ΔH_{cal} by 59 kcal/mol in the presence of 0.1% (w/v) SDS. Further increase in the detergent concentration to 0.2% led to a further lowering in Tₘ by 3° C. and AHca, by 41 kcal/mol (Table 7). CD scan of the two enzymes in the presence of 0.1-1% (w/v) SDS further revealed no alteration for AL protease while complete loss of secondary structure was seen for Subtilisin Carlsberg (data not shown).

TABLE 7
Effect of SDS on thermodynamic parameters for AL protease and
Subtilisin Carlsberg at pH 10.0.

| Enzyme | Tₘ (° C.) | ΔH_{cal} (kcal/mol) |
|---|---|---|
| AL, untreated | 74.5 ± 0.03 | 158 ± 0.6 |
| +0.1% SDS | 74.5 ± 0.03 | 158 ± 0.7 |
| +0.5% SDS | 74.1 ± 0.02 | 158 ± 0.7 |
| +1% SDS | 73.1 ± 0.02 | 154 ± 0.6 |
| Subtilisin, | 67.3 ± 0.02 | 203 ± 0.9 |
| untreated | | |
| +0.1% SDS | 54.5 ± 0.02 | 144 ± 0.5 |
| +0.2% SDS | 51.1 ± 0.01 | 103 ± 0.2 |

Effect of oxidation by H₂O₂ on AL protease stability was determined by residual activity measurements because of the temperature-induced decomposition of the peroxide in the calorimeter. As shown in FIG. 13, H₂O₂ was required at rather high concentrations for the enzyme activity to be affected. Incubation with 1 M peroxide at 40° C. for 90 min reduced the activity to 60% of the original, and 1.6 M peroxide did not reduce the activity further. Residual activity of the AL protease was also studied after incubation with sodium tripolyphosphate (STP; Sigma), sodium-Zeolite 4A (Prayon Tessenderlo, Belgium) and sodium citrate, respectively, representing some of the sequestering agents used in washing detergents. The enzyme activity was unchanged in the presence of 0.01-0.05% (w/v) Zeolite and STP, respectively, after 90 min at 40° C., and more than 80% of the original activity was retained in the presence of 0.5 M citrate (data not shown).

Protein engineering using site-directed or random mutagenesis has been the main tool for Improving the stability of subtilisin type proteases. At the same time, however, a number of naturally occurring alkaline proteases, mostly from Bacillus sp. but also from other sources, have been reported having stability with respect to one or more of the conditions/additives encountered during washing with detergents (Samal, B B et al (1990). Alkaliphilic Bacillus species have provided a series of commercial proteases, e.g. Esperase™ and Savinase™, having greater alkali tolerance than the subtilisins from other sources.

This work analyses the stability characteristics of an alkaline protease from another alkaliphilic micro organism, Nesterenkonia sp. AL. The enzyme has a molecular weight of 22876±5 Da (determined using MALDI-TOF mass spectrometry), isoelectric point of 4.2, and possesses optimal activity at pH 10.0. Being one of the standard commercial enzymes, Subtilisin Carlsberg was used as a model for comparison in this study. Calorimetry studies showed the AL protease to possess higher thermo stability than subtilisin (FIG. 12). Moreover, the AL enzyme did not require the presence of a protease inhibitor during monitoring of thermal denaturation by DSC, which is normally the case with other proteases, as seen for subtilisin, due to the autolytic digestion of the enzymes. The unfolding transition of AL protease occurred by a two-state process under all conditions, as suggested by symmetric thermograms and equal ΔH_{cal} and ΔH_{vH} values. The thermodynamic stability was maintained over a range of pH conditions, and in the presence of significantly high concentrations of surfactant and metal chelator. CD scan of the enzyme also confirmed that the secondary structure of the enzyme remains intact under various stress conditions (FIG. 11).

A universal feature among the subtilisin type proteases is the presence of one or more calcium binding sites, which make large contributions to their stability against thermal denaturation and autolytic digestion. Proteases with higher thermo stability exhibit enhanced calcium binding. One of the sites invariably has significantly lower association constant for the calcium Ions than the other(s), and removal of just the weakly bound calcium destabilizes the enzyme significantly. A drastic reduction in melting temperature of Subtilisin Carlsberg was observed in the presence of EDTA (FIG. 12b), which was also the case with subtilisin BPN′. Calcium binding is an excellent strategy to allow survival of the enzymes in the extracellular environment but poses a limitation during use of the enzymes in detergents that contain sequestering agents to complex calcium and magnesium—the water hardness ions. Hence, calcium-independent variants have been obtained, e.g. by removal of the calcium-binding loop and subsequent directed mutagenesis. Engineering a disulfide bond in the calcium free subtilisin has also led to improved stability of the enzyme.

AL protease was found to possess negligible amount (about 0.143 mol/mol) of calcium associated to It. This may most likely indicate that the enzyme has no calcium-binding site, and hence its conformation and activity were influenced neither by the presence of the metal ion nor the metal-binding compounds. The AL protease structure has evolved to adapt to the soda lake environment from which the organism was isolated. The lakes have a pH of 10.0-11.0 and as a consequence of high carbonate/bicarbonate concentration, calcium and magnesium concentrations are very low due to formation of insoluble salts.

A methionine residue at position 222 next to the active site is another conserved feature among Bacillus subtilisins, which is susceptible to oxidation and restricts their industrial utility. Bleaching compounds such as perborate or percarbonate, present in detergent formulations to ensure good wash performance, generate hydrogen peroxide when dissolved in water. Production of methionine sulfoxide in the presence of H₂O₂ is correlated to loss of activity and decreased thermal stability of the enzyme. Substitution of the methionine with other non-oxidizable amino acids has been used to produce oxidation-resistant variants, however with compromised catalytic activity. AL protease exhibited good activity retention at very high levels of H₂O₂ (FIG. 13), again suggesting a difference at the molecular level from the subtilisins. It has previously been reported another alkaline protease from Bacillus sp. exhibiting high resistance to oxidation, the structure of which is not yet reported.

Crystallization and X-Ray Analysis of AL

A large body of information has been assembled on the crystal structure and biological function relationship of several members of the subtilisin family. They all possess similar arrangement of catalytic triad consisted of Ser, His and Asp residues in an α/β protein scaffolds (Siezen & Leunissen, 1997). Another common feature is the presence of one or more calcium binding sites. The bound calcium plays an important role in providing stabilization against thermal denaturation and autolytic digestion; enhanced calcium binding is thus observed for the proteases with higher thermo stability (Bryan et al., 1986; Frömmel & Höhne, 1981; Strausberg et al., 1995). The subtilisin family of serine proteases has been subjected to extensive enzymology, structure and engineering studies for understanding their catalytic mechanism, specificity, stability and also for improving their function for industry applications.

### Experiment 5

Crystallization

Materials and Methods 5

The alkaline protease was purified from the culture supernatant of the Nesterenkonia sp. AL according to the procedure described earlier, comprising ammonium sulphate precipitation, ion exchange chromatography and gel filtration. The purified AL protease was obtained with a recovery of 57% and an overall purification factor of 48-fold, having a specific activity of about 2200 U mg⁻¹. Fractions containing the enzyme in 10 mM Tris-HCl buffer, pH 8.0 were pooled and concentrated to 2.8 mg ml⁻¹ for crystallization.

The initial crystallization conditions were screened by the sparse-matrix method (Jancarik & Kim, 1991). All crystallization experiments were performed by the hanging-drop vapour diffusion method using Hampton Research crystallization kits II, and I at 294 K in 24-well VDX plates (Hampton Research, USA). In each trial, a hanging drop of 1 μl of protein solution was mixed with 1 μl of reservoir solution, and equilibrated against 500 μl of the reservoir solution. The additive screens (Hampton Research, USA) were also tried to optimise the crystallization conditions.

An AL protease crystal with dimensions of about 0.5×0.3×0.3 mm was briefly soaked with cryo-protectant (1100% reservoir solution with 5% glycerol), picked up by a nylon CryoLoop (Hampton Research, USA), and plunged into liquid nitrogen directly. The frozen crystal was transferred to a cold nitrogen stream (Oxford Cryostream Cooler) operated at 100 K. Diffraction data were collected at the crystallographic beamline BL711 at the MAX-II synchrotron laboratory in Lund (Sweden) by the oscillation method with wavelength of 1.076 Å, using a MarCCD detector (X-ray Research, GbmH.). The crystal-to-detector distance was 80 mm, with the oscillation range of 0.5° per image. The exposure time was typically 60 seconds per frame. The diffraction data were processed by the program XDS and merged with XSCALE. The data collection statistics is listed in Table 8.

TABLE 8
Data collection statistics.

| | |
|---|---|
| Wavelength (Å) | 1.076 |
| Resolution (Å) | 30.0-1.39 |
| | (1.47-1.39)* |
| Completeness (%) | 97.4 (90.0) |
| R_{merge} (%)^{\} | 4.9 (19.3) |
| I/σ(I) | 13.7 (4.3) |
| Space group | R3 |
| Cell parameters (Å) | a = 92.26 |
| | b = 92.26 |
| | c = 137.88 |
| | γ = 120.0 |
| Possible no. of unique reflections | 88 606 |
| Total No. of reflections collected | 283 174 |
| Vₘ (Å³/Da) | 2.68 |
| Molecules per asymmetric unit | 2 |

*Values in parenthesis are for the last resolution shell.
^{\}R_{merge} = Σ|I_{obs} − I_{avg}|/ΣI_{obs} where the summation is over all reflections.

Initial screening of the crystallization conditions showed thin, needle-like crystals in drop number 33 of Crystal Screen I (4.0 M sodium formate only). This condition was further optimised by fine screening of the sodium formate and protein concentrations, buffer content, 10 and the pH values. The addition of 3% (v/w) ethanol, 0.01 M L-cysteine or 0.01 M NAD⁺ seemed to improve crystal quality of this protein.

As shown in FIG. 14, triangular-prism shaped crystals could be obtained with typical dimensions of 0.2×0.1×0. 1 mm in 2.9 M sodium formate, with 0.1 M sodium citrate, 15 pH 6.9 during 4-5 days at room temperature. The macro-seeding procedure using a nylon CryoLoop could produce large and nice-looking single crystal. The crystals grown around such condition are very reproducible particularly with macro-seeding. Some crystals of AL diffracted to beyond 1.5 A resolutions on beamline BL711 of the MAX-II synchrotron in Lund.

A complete data set of 1.39 A resolution was collected and processed as summarized in Table 8. The crystal belongs to the space group R3, with unit cell parameters a=b=92.26 Å, c=137.88 Å, y=120°. Most likely, there are 2 molecules per asymmetric unit as estimated by the calculated V_{M} (Matthews, 1968) value of 2.68 Å³.Da⁻¹. The solvent content for such a crystal is then approximately 54%. Self-rotation function was calculated on the scaled data (resolution range 15-4 Å was used for calculation) from the native crystal using the program Polarrfn with the CCP4 program suite (CCP4, 1994). At κ=180° section, it was shown that there are three pairs of NCS (non-crystallographic symmetry) 2-fold axes related by the crystallographic three-fold symmetry. This 2-fold NCS fits well with above estimated two molecules per asymmetric unit.

Determination of the primary sequence of the AL alkaline protease has been carried out, and the structure solution is also progressing by MIR (multiple isomorphous replacement) methods. Some derivatives have been prepared by soaking native protein crystals in different heavy atom compounds; some of the soaked crystals seem to diffract well, and to contain heavy metal sites.

Enzymes are normally incorporated Into detergent or detergent additive compositions at levels sufficient to provide a “cleaning-effective amount”. The term “cleaning effective amount” refers to any amount capable of producing a cleaning, stain removal, soil removal, whitening, deodorizing, or freshness improving effect on substrates such as dishware, clothes and the like. In practical terms the compositions herein may comprise from 0.001% to 5%, preferably 0.01%-1% by weight of an enzyme preparation of the present invention. Protease enzymes are usually present in such commercial preparations at levels sufficient to provide from 0.005 to 0.1 Anson units (AU) of activity per gram of composition.

### EXAMPLE

Detergent compositions were prepared according to the tables given below, which compositions contained the alkaline protease of the present invention derived from Nesterenkonia sp. nov. AL.

| | Detergent | Detergent | Detergent |
|---|---|---|---|
| Compound | A | B | C |
| Alkaline protease | 0.5 | 0.5 | 0.5 |
| acc. to invention | | | |
| Sodium linear alkyl | 23.0 | 4.0 | 20.0 |
| (C12-C14) benzene | | | |
| sulfonate | | | |
| Alkyl sulfate | 4.0 | | |
| Polyoxyethylene | 5.0 | | |
| lauryl ether) | | | |
| Polyoxyethylene-polyoxypropylene | | 5.0 | |
| lauryl ether | | | |
| Alkyl ether sulfate | | 20.0 | |
| Fatty acid salt | 3.0 | 2.5 | 2.0 |
| Zeolite | 21.5 | | 20.0 |
| Sodium carbonate | 15.0 | | 15.0 |
| Potassium carbonate | 3.0 | | 1.5 |
| Amorphous silicate | 7.0 | | 7.0 |
| Crystalline silicate | 4.0 | | |
| Sodium sulfite | 2.0 | 0.5 | 2.0 |
| Sodium sulfate | 2.0 | | 23.0 |
| Acrylic acid- | 5.0 | | |
| maleic acid copolymer | | | |
| Citrate | | 10.0 | |
| PEG | 2.0 | | 2.0 |
| Monoethanolamine | | 8.0 | |
| Ethanol | | 5.0 | |
| Water | 3.0 | 45.5 | 7.0 |
| Form | Gran. | Liq. | Gran. |

Polyoxyethylene lauryl ether (average ethylene oxide addition is 4 moles)
Polyoxyethylene-polyoxypropylene lauryl ether (average ethylene oxide addition is 8 mol, and average propylene oxide addition is 3 mol)
Alkyl ether sulfate (average ethylene oxide addition is 2.5 mol)
Fatty acid sodium salt
Zeolite: average particle size of 2-3 μm
Amorphous silicate: sodium silicate
Crystalline silicate: SKS-6, average particle size 10-15 μm
Acrylic acid-maleic acid copolymer (Sokalan CP5 product of BASF)
PEG: polyethylene glycol, average molecular weight is 8,000

The detergents produced below are useful for a laundry detergent.

| | Detergents of the present invention (% w/w) | | | | | | |
|---|---|---|---|---|---|---|---|
| Component | D | E | F | G | H | J | K |
| Alkylbenzene | 20 | | 20.5 | 12 | 20 | | 10 |
| sulfonic acid | | | | | | | |
| sodium salt | | | | | | | |
| Alkylbenzene | | 15 | | | | | |
| sulfonic acid | | | | | | | |
| sodium salt | | | | | | | |
| Sodium alkane | 3 | | | | | | |
| sulfonate | | | | | | | |
| Sodium olefin | | 3 | | | | | |
| sulfonate | | | | | | | |
| Sulfo-fatty acid | | 8 | | | | | |
| methyl ester | | | | | | | |
| Sodium | 2 | 6 | 4 | 3 | 2 | 1.5 | |
| palmitate | | | | | | | |
| Ethylene | 3 | 3 | 3 | | 5 | 40 | 30 |
| oxide adduct | | | | | | | |
| Alkyl glucoside | | | | | | | 5.4 |
| Zeolite | 30 | 15 | 15 | | 20 | | |
| Amorphous | 12 | 1 | 8 | 10 | 5 | | |
| silicate | | | | | | | |
| Sodium tripoly- | | | | 25.5 | | | |
| phosphate | | | | | | | |
| Sodium | 10 | 23.5 | 24.5 | | 17.5 | 0.5 | |
| carbonate | | | | | | | |
| Potassium | | 3 | | 5 | | | |
| carbonate | | | | | | | |
| Sodium | 2 | 2 | | 1 | | 0.2 | 0.2 |
| sulfite | | | | | | | |
| Sodium | 5 | 1.5 | | 9.5 | 10 | | |
| sulfate | | | | | | | |
| Sodium | | 4 | | 5 | 1.5 | 1.5 | 1 |
| citrate | | | | | | | |
| Monoethanol | | | | | | 4 | 5 |
| amine | | | | | | | |
| Polyacrylic | | | | 1 | 3 | | |
| acid | | | | | | | |
| Acrylic acid- | | 3 | 3 | 5 | | | |
| maleic acid | | | | | | | |
| copolymer | | | | | | | |
| CMC | 2 | | | | | | |
| PEG | 5 | 2 | 2 | 2 | 2 | 1.5 | |
| PVP | | | | 2 | | | |
| Enzyme | 2 | 2 | 2 | 3 | 2 | 0.1 | 0.2 |
| Water | 4 | 5 | 3 | 1 | 5 | 43.7 | 38.2 |
| Ethanol | | | 5 | 5 | 5 | 5 | 5 |
| Propylene | | | | | | 2 | 5 |
| glycol | | | | | | | |
| Na-percarbonate | | 3 | 10 | | | | |
| Total | 100 | 100 | 100 | 100 | 100 | 100 | 100 |
| Form | G | G | G | G | G | L | L |

Bleaching agents for laundry.

Bleaching Agents of the Present Invention

| Component | M | N | O | P |
|---|---|---|---|---|
| Sodium percarbonate¹⁾ | 80.0 | 80.0 | 80.0 | 80.0 |
| Sodium carbonate | 16.0 | 12.0 | 16.0 | 12.0 |
| Anionic surfactant²⁾ | 2.0 | 2.0 | — | — |
| Nonionic surfactant³⁾ | — | — | 2.0 | 2.0 |
| Sodium polyacrylate⁴⁾ | 1.0 | 1.0 | 1.0 | 1.0 |
| Sodium lauroyloxy- | — | 4.0 | — | 4.0 |
| benzene sulfonate | | | | |
| Alkaline protease | 1.0 | 1.0 | 1.0 | 1.0 |

¹⁾Particle size: 500-700 μm
²⁾Sodium linear alkylbenzene sulfonate
³⁾Polyoxyethylene alkyl ether (C12-C14 alkyl), ethylene oxide addition is 12 mol
⁴⁾Average molecular weight of 8,000

Detergents for a dishwasher.

Detergents of the Present Invention

| Component | Q | R | S | T |
|---|---|---|---|---|
| Pluronic L-61¹⁾ | 4 | — | 4 | 4 |
| Softanol EP-7085²⁾ | — | 4 | — | — |
| Trisodium citrate | 30 | 30 | — | — |
| EDTA | — | — | 30 | — |
| Sodium tripolyphosfate | — | — | — | 30 |
| Sodium percarbonate | 20 | 20 | 20 | 20 |
| Sodium carbonate | 20 | 20 | 20 | 20 |
| (dense ash) | | | | |
| Amorphous silicate³⁾ | 10 | 10 | 10 | 10 |
| A-M* | 4 | 4 | 4 | 4 |
| Sodium sulfate | 10 | 10 | 10 | 10 |
| Lipolase 100T ® | 0.5 | 0.5 | 0.5 | 0.5 |
| (Novo Nordisk) | | | | |
| Termamyl 60T ® | 1 | 1 | 1 | 1 |
| (Novo Nordisk) | | | | |
| Alkaline protease | 0.5 | 0.5 | 0.5 | 0.5 |

¹⁾Polyoxyethylene-polyoxypropylene copolymer (mw 2,000)
²⁾Ethylene oxide (7 moles) and propylene oxide (8.5 moles) adduct of C12-C14 sec-alcohol
³⁾Sodium silicate
*Acrylic acid-maleic acid copolymer

Detergent Compositions of the Present Invention

| Component | U | V | W | X | Y |
|---|---|---|---|---|---|
| Sodium carbonate | 30 | | 30 | | 50 |
| Sodium hydrogen- | | 25 | | 25 | |
| carbonate | | | | | |
| Sokalan CP5¹⁾ | 5 | 6 | 5 | 5 | 5 |
| Sodium hydrogen- | 5 | | 6 | | |
| percarbonate | | | | | |
| Limonene | 2 | 2 | | 1 | 1 |
| Softanol EP7045²⁾ | | | 2 | 1 | 1 |
| Amorphous sodium | 2 | | 2 | 1 | 3 |
| aluminosilicate | | | | | |
| Amorphous sodium | | 2 | | 1 | |
| aluminosilicate | | | | | |
| Lipolase 100T ® | 0.5 | 0.5 | 0.5 | 0.5 | 0.5 |
| (Novo Nordisk) | | | | | |
| Termamyl 60T ® | 1 | 1 | 1 | 1 | 1 |
| (Novo Nordisk) | | | | | |
| alkaline protease | 0.5 | 0.5 | 0.5 | 0.5 | 0.5 |
| Sodium malate | | 10 | | 5 | |
| Sodium citrate | 15 | | 10 | 4 | 8 |
| Sodium sulfate | 39 | 53 | 43 | 55 | 30 |

¹⁾Acrylic acid-maleic acid copolymer (product of BASF)
²⁾Ethylene oxide (7 moles) and propylene oxide (4.5 moles) adduct of C12-C14 sec-alcohol

Liquid Enzyme Composition for the Soaking of Hides and Skins

In a vessel, 300 g of a nonionic surfactant made of C₁₃-oxoalcohol condensed with 6 moles of ethylene oxide, 682 g of a nonionic surfactant made of C₁₃-oxoalcohol and 9 mol of ethylene oxide, 13 g of Aerosil® 380 (fumed silica, product of Degussa A G, W.-Germany), and 5 g of Borchigen® STL a surface active agent are dispersed with a toothed-disk agitator at a rotative speed of 15-18 m/s for 15 minutes. Then 27.5 g of an alkaline protease from Nestenkornia sp. nov. AL (activity 1800 U/mg) and 10 g of a fungal protease from Aspergillus parasiticus (activity 220 000 U/g) are added in this order and are dispersed for another 10 minutes. One obtains a liquid, homogenous enzyme preparation which can be easily transferred by pouring or pumping and which has not settled or creamed after 4 weeks. The activity after this period of time at 15° C. is 96% of the original one, which was 4 400 U/g.

The Nesterenkonia sp. nov. AL is a stable alkaline serine protease useful in industrial and enzymatic cleaning applications. This product has excellent temperature stability and is resistant to attack from chelating reagents and surfactants- either ionic or non-ionic.

The protease of the invention is a versatile product for many industrial reactions and processes. Examples include silver recovery from x-ray films, industrial machinery cleaning and maintenance applications, detergent and pre-spotter formulations and, other uses where broad spectrum proteolytic activity is called for.

### ABBREVIATIONS

N-succinyl-Leu-Tyr-7-amino-4-methyl coumarin (Suc-L-Y-AMC), N-succinyl-Ala-Ala-Phe-7-amino-4-methyl coumarin (Suc-A-A-F-AMC), N-succinyl-Ile-Ile-Trp-7-amino-4-methyl coumarin (Suc-I-I-W-AMC), N-succinyl-Ala-Ala-Lys-7-amino-4-methyl coumarin (Suc-A-A-K-AMC), NA-benzoyl-L-Arg-7-amino-4-methyl coumarin (NA-Bz-R-AMC), N-succinyl-Leu-Leu-Val-Tyr-7-amino-4-methyl coumarin (Suc-L-L-V-Y-AMC), N-succiny-Phe-Gly-Leu-β-naphthylamide (Suc-F-G-L-β-NNap), N-succiny-Leu-β-naphthylamide (Suc-L-β-NNap), N-benzoyl-L-tyrosine-p-nitroanilide (Bz-Tyr-pNA), Nα-benzoyl-D,L-arginine-p-nitroanilide (BAPNA), N-succinyl-phenylalanine-p-nitroanilide (Suc-Phe-pNA), N-succinyl-Ala-Ala-Pro-Phe-p-nitroanilide (Suc-A-A-P-F-pNA), N-succinyl-Ala-Ala-Pro-Leu-p-nitroanilide (Suc-A-A-P-L-pNA), N-succinyl-Gly-Gly-Gly-p-nitroanilide (Suc-G-G-G-pNA), N-Succinyl-Ala-Ala-Ala-p-nitroanilide (Suc-A-A-A-pNA), N-succinyl-Ala-Ala-Val-p-nitroanilide (Suc-A-A-V-pNA), S-benzyl-L-cysteine-4-nitroanilide, N_{α}-benzoyl-L-arginine ethyl ester (BAEE), N-benzoyl- L-tyrosine ethyl ester (BTEE), N-benzoyl-D, L-phenylalanine β-naphthyl ester (Bz-F-NE), p-nitrophenyl acetate, p-nitrophenyl butyrate and N-succinyl-Phe-Leu-Phe thiobenzyl ester (Suc-F-L-F-SBzl).

### LEGENDS TO FIGURES

FIG. 1. Scanning electron photomicrograph showing the morphology of the strain Nesterenkonia sp. nov. AL. Bar 1 μm.

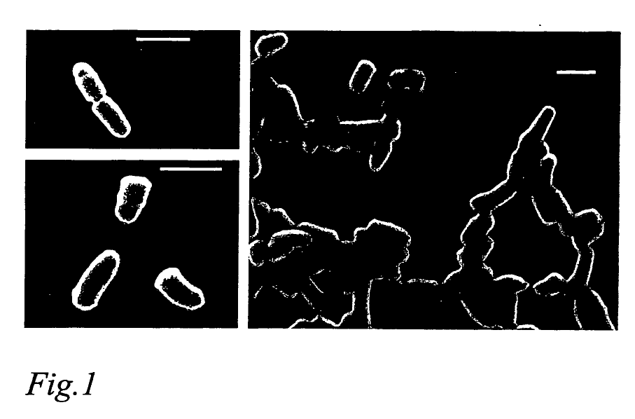

FIG. 2. Phylogenetic tree derived from analysis of the 16S rDNA sequences of AL strain and related species belonging to the branch of gram-positive bacteria with a high G+C content. The accession numbers of the sequences of the strain used in the phylogenetic analyses are: DSM 15380, AL; Y13857, Nesterenkonia halobia RH-59; X83405, Arthrobacter aurescens DSM 20116; X80737, A. citreus DSM 20133; X80738, A. crystallopoietes DSM 20117; X80736, A. globiformis DSM 20124; X83407, A. ilicis DSM 20138; X80739, A. nicotianae DSM 20123; X83408, A. oxydans DSM 20119; X80740, A. pascens DSM 20545; X80745 A. protophormiae DSM 20168; X80742, A. ramosus DSM 20546; X83410, A. uratoxidans DSM 20647; X80744, A. ureafaciens DSM 20126; X80749, Kocuria kristinae DSM 20032; X87756, K. rosea DSM 20447; X87754, K. varians DSM 20033; X80748, Microcuccus agilis DSM 20550; M38242, M. luteus ATCC 381; X80750, M. Iylae DSM 20315; X80747, N. halobia DSM 20541; M59055, Rothia dentocariosa ATCC 17931; X87758, Stomatococcus mucilaginosus DSM 20746.

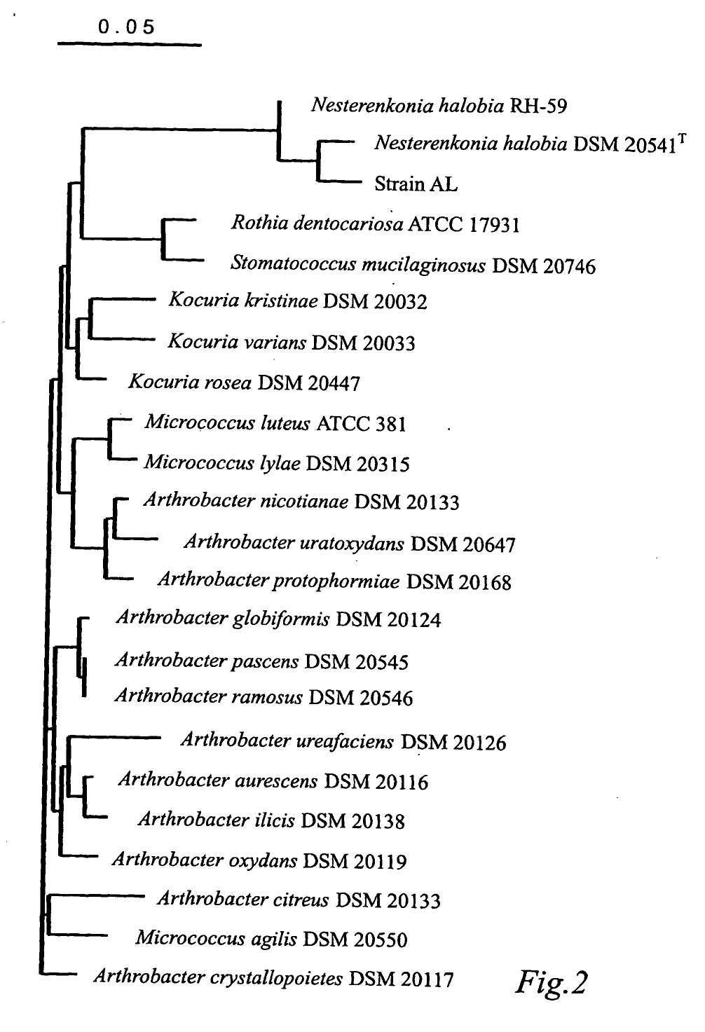

FIG. 3. MALDI spectra of hydrolysis products of oxidized insulin B-chain. Proteolysis was carried out at 50° C. with an enzyme-substrate ratio of 1:100 in 10 mM Tris-HCl buffer, pH 9, (A) for 15 min (B) for 12 h.

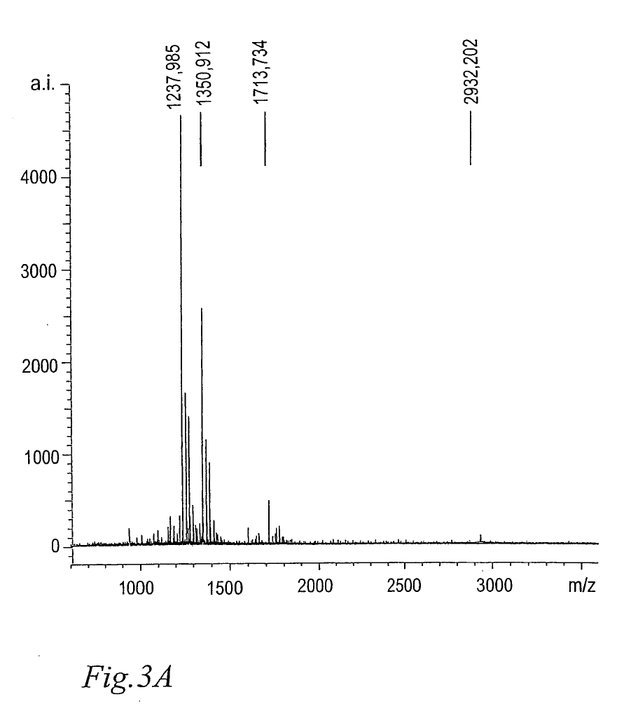

FIG. 4. Initial cleavage site of oxidized insulin B-chain by the AL protease. Solid lines indicate the preliminary cleavage sites and dashed lines indicate the completed hydrolysis.

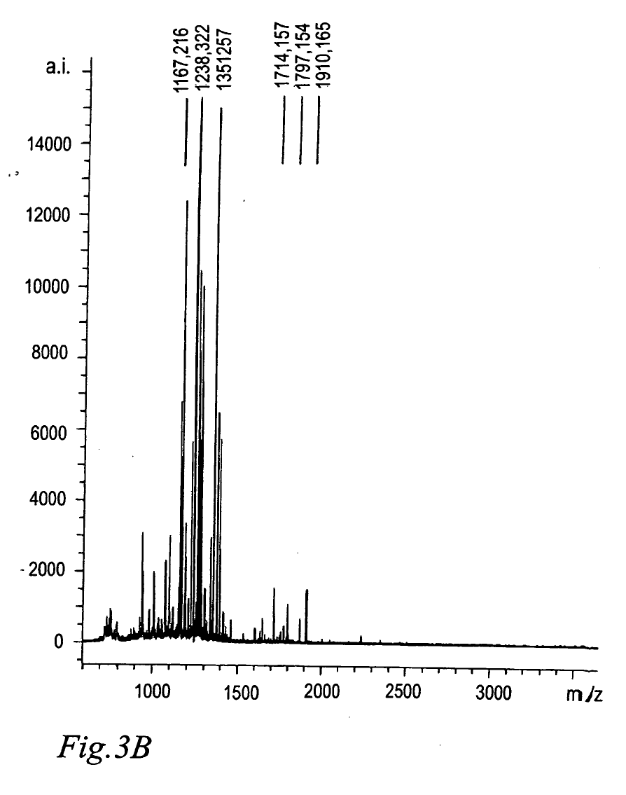

FIG. 5. Hydrolysis of keratin-azure (10 mg) by 5 mg of the AL protease in 10 mM glycine-NaOH, pH 10 at 50° C. for 24 h period.

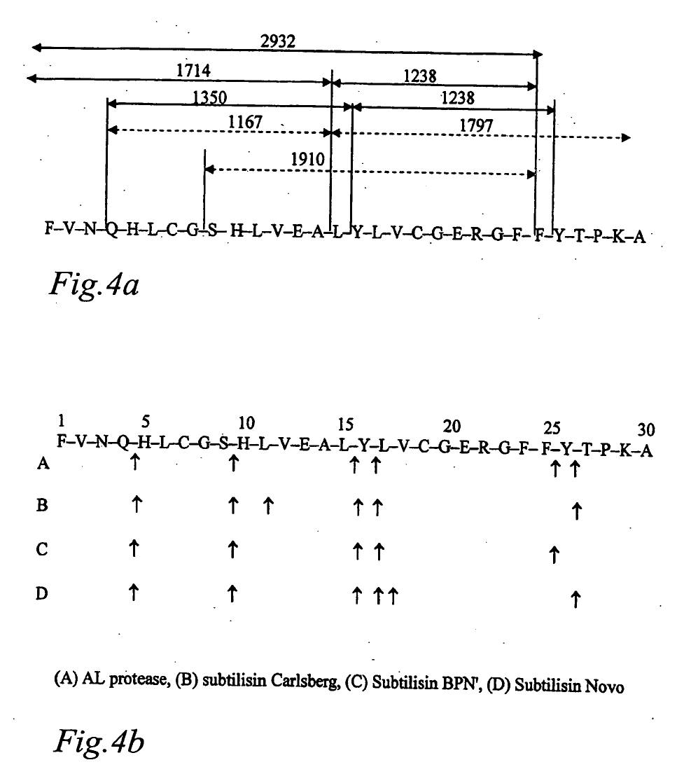

FIG. 6. Hydrolysis of hemoglobin (□) and casein (·) by AL protease in 50 mM glycin-NaOH, pH 10 at 50° C. for 20 min.

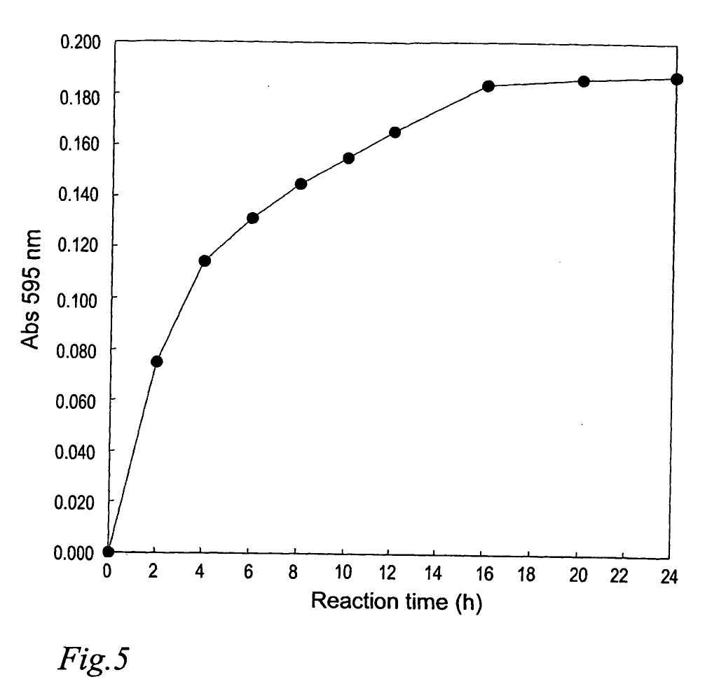

FIG. 7. Effect of pH on the activity of (A) AL protease and (B) AL-89 protease. The protease activity was assayed at 50° C. Buffers used were phosphate, pH 6-8 (●); Tris-HCl, pH 7.5-9 (▪); and glycine-NaOH, pH 8.5-12 (▴), each at a concentration of 50 mM. The data points are average of 3 separate measurements.

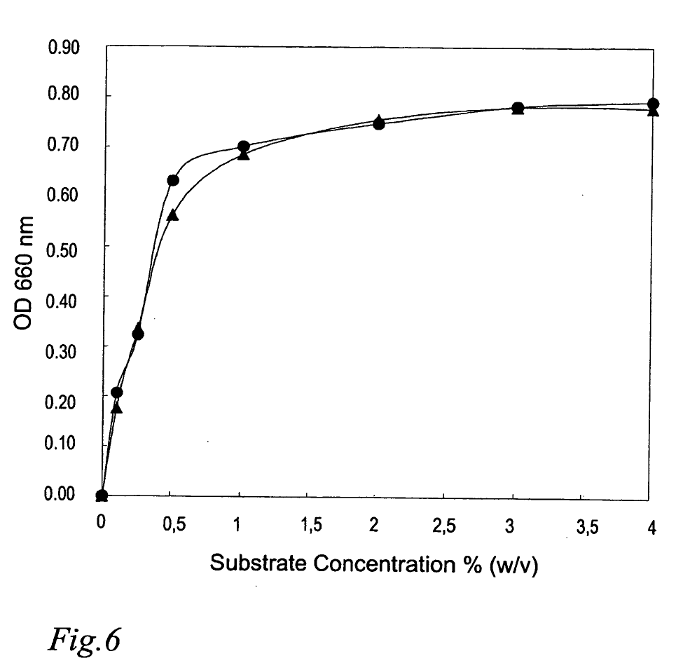

FIG. 8. Effect of pH on the stability of (A) AL protease and (B) AL-89 protease. The enzyme was incubated in different buffers at 50° C. for 1 h and residual activity was measured using standard assay procedure. Buffers used were: citrate-phosphate, pH 4-7 (●), phosphate, pH 6-8 (▪), Tris-HCl, pH7.5-9.0 (▴), and glycine NaOH, pH 8.5-11 (▾), each at a concentration of 50 mM. The data points are average of 3 separate measurements.

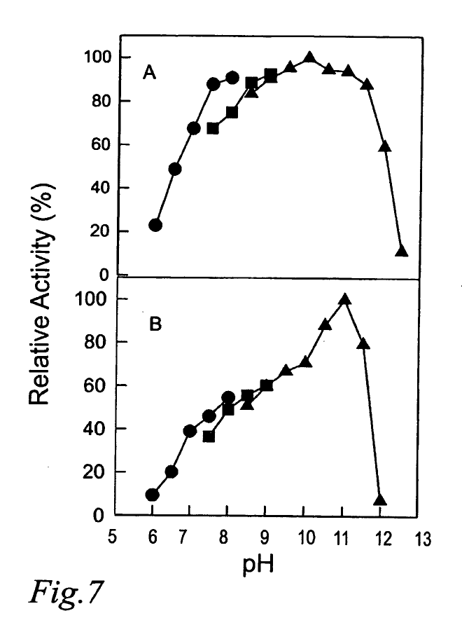

FIG. 9. Temperature profile of (A) AL protease and (B) AL-89 protease assayed in the presence (●) and absence (▪) of 5 mM Ca²⁺. The data points are average of 3 separate measurements.

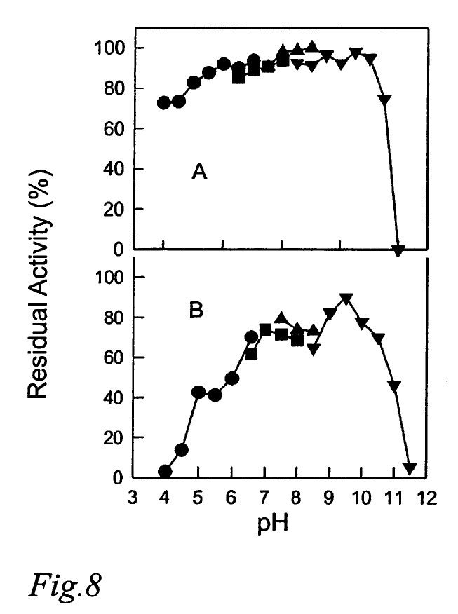

FIG. 10. Thermal stability of (A) AL protease and (B) AL-89 protease determined in the absence (open symbols) and presence (closed symbols) of 5 mM Ca²⁺. The enzymes were incubated at 55° C. (◯, ●), 60° C. (□, ▪), 65° C. (Δ, ▴), and 70° C. (∇, ▾), respectively, in 50 mM glycine NaOH buffer, pH 10. Samples were withdrawn at definite time intervals and residual activity was measured following standard assay procedure. The data points are average of 3 separate measurements.

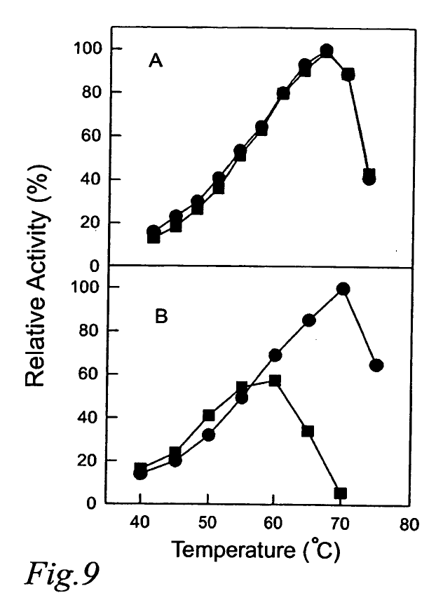

FIG. 11. Circular dichroism scan of (a) AL protease and (b) Subtilisin Carlsberg before (---) and after (-) storage for 24 h at 50° C. in 10 mM glycine-NaOH buffer, pH 10.0. The concentration of AL protease and Subtilisin Carlsberg was 150 μg/ml.

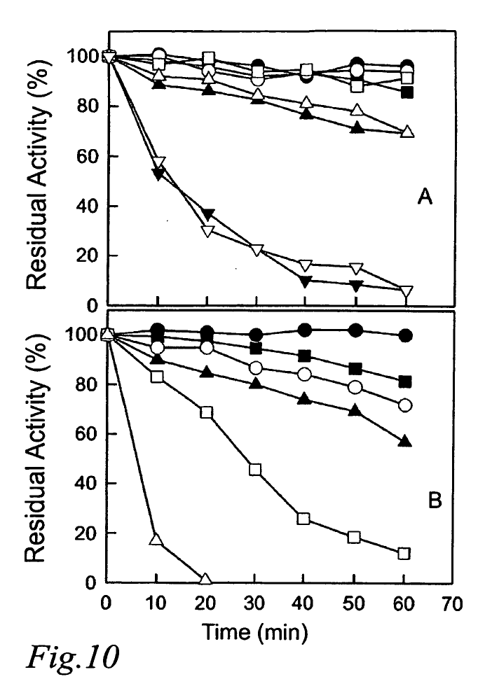

FIG. 12. Differential scanning calorimetry scans of (a) AL protease and (b) Subtilisin Carlsberg in 10 mM glycine-NaOH buffer, pH 10.0. The samples were untreated (-), chelex treated (Δ); and in the presence of 5 mM CaCl₂ ( . . . ), and 10 mM EDTA (---), respectively. The concentrations of AL and subtilisin were 0.3 mg /ml and 2.5 mg/ml, respectively.

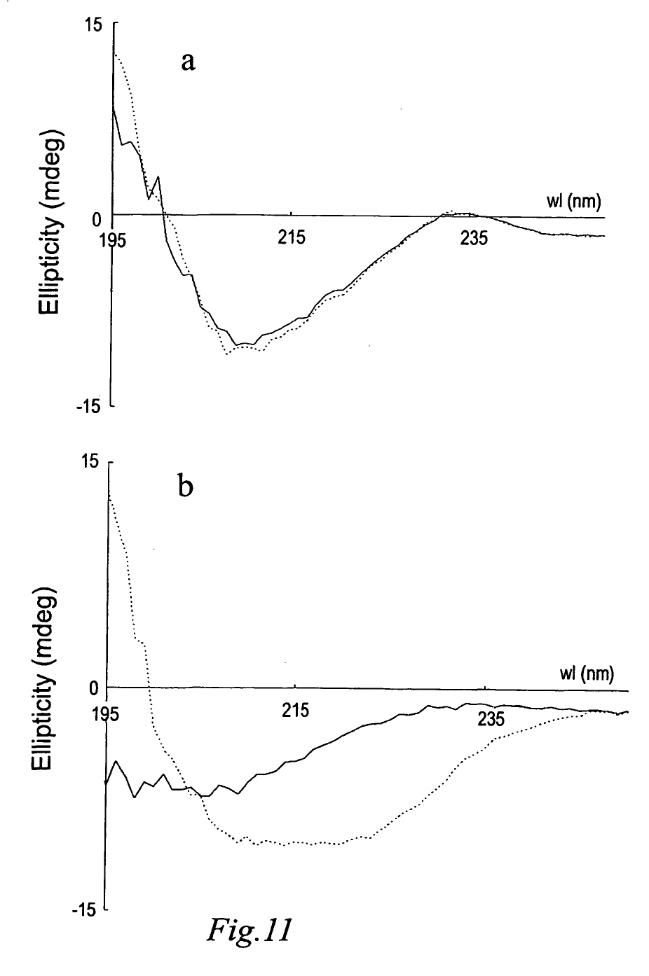

FIG. 13. Residual activity of AL protease in the presence of 0.1(Δ), 1.0 (□) and 1.6 M (◯) hydrogen peroxide in 10 mM glycine-NaOH, pH 10.0 at 40° C. The data represent an average of three measurements. Standard deviation of the measurements is indicated by error bars.

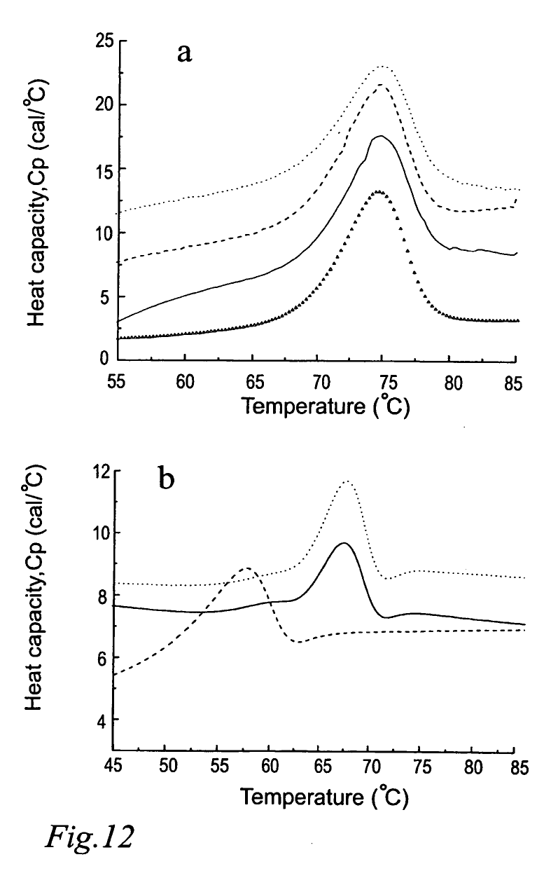

FIG. 14. Triangular-prism shaped crystals of AL alkaline protease grown in 2.9 M sodium formate, 0.1 M sodium citrate, pH 6.9 at room temperature; these crystals can be used as seeds to get even larger crystals.

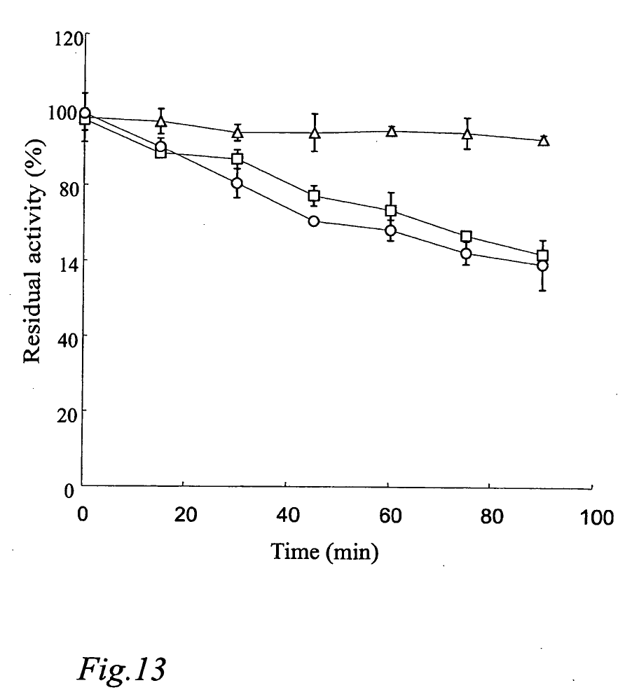

FIG. 15 The self-rotation function of AL protease crystal calculated between 15-4 Å resolution and plotted at κ=180° section.

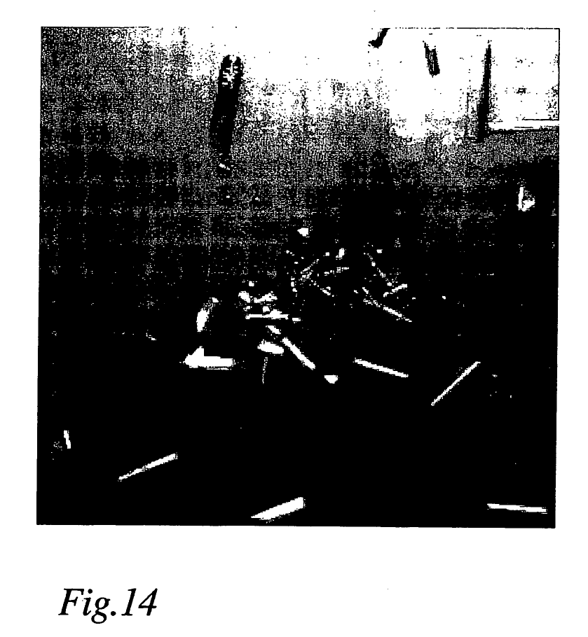

### REFERENCES

Anwar, A., and M. Saleemuddin. 1998. Alkaline proteases: A review. Bioresour Technol 64: 175-183.
Banerjee, U. C., R. K. Sani, W. Azmi, and R. Soni. 1999. Thermostable alkaline protease from Bacillus brevis and its characterization as a laundry detergent additive. Process Biochemistry 35: 213-219.
Bott, R. Development of new proteases for detergents. In: Enzymes in Detergency. Surfactant science series, 1997; vol. 69, pp 61-74, van Ee, J. H., Misset, O. and Baas, E. J. (eds), Marcel Dekker, New York.
Bryan, P. N., Rollence, M. L., Pantoliano, M. W., Wood, J., Fizel, B. C., Barry, C. F., Gilliland, G. L., Howard, A. J. & Poulos, T. L. (1986). Proteins: Struct. Funct. Genet. 1, 326-334.
Cashion, P., Holder-Franklin, M. A., McCully, J. & Franklin, M. (1977). A rapid method for the base ratio determination of bacterial DNA. Anal Biochem 81, 461-466.
Duckworth, A. W., Grant, W. D., Jones, B. E. & van Steenbergen, R. (1996). Phylogenetic diversity of soda lake alkaliphiles. FEMS Microbiol Ecol 19, 181-191.
Duckworth A. W., Grant, W. D., Jones, B. E., Meijer, D., Marquez, C., & Ventosa, A. (2000). Halomonas magadii sp. nov., a new member of the genus Halomonas, isolated from a soda lake of the East African Rift Valley. Extremophiles 4, 53-60.
De Ley, J., Cattoir, H. & Reynaerts, A. (1970). The quantitative measurement of DNA hybridization from renaturation rates. Eur J Biochem 12, 133-142.
Egmond M R. Application of proteases in detergents. In: Enzymes in Detergency. Surfactant science series, 1997; vol. 69, pp 61-74, van Ee J H, Misset O, Baas E J (eds), Marcel Dekker, New York.
Escara, J. F. & Hutton, J. R. (1980). Thermal stability and renaturation of DNA in dimethylsulphoxide solutions: acceleration of renaturation rate. Biopol 19, 1315-1327.
Ferrero M A, Castro G R, Abate C M, Baigori M D, Slneriz F. Thermostable alkaline proteases of Bacillus licheniformis MIR 29: isolation, production and characterization. Appl Microbiol Biotechnol 1996;45:327-32.
Frömmel, C. & Höhne, W. E. (1981). Biochim Biophys Acta 670, 25-31.
Gregg, K., S. D. Wilson, D. A. D. Parry, and G. E. Rogers. 1984. A comparison of genomic coding sequences for feather and scale keratins: Structural and evolutionary implications. EMBO J 3:175-178
Gupta, R., K. Gupta, R. K. Saxena, and S. Khan. 1999. Bleach-stable, alkaline protease from Bacillus sp. Biotechnol. lett 21: 135-138.
Horikoshi, K. 1999. Alkaliphiles: some application of their products for biotechnology. Microbiol. Mol. Biol. Rev 63: 735-750.
Horikoshi, K. (1998). Al kaliphiles. In Horikoshi, K. & Grant, W. D., Eds., Extremophiles: Microbial Life in Extreme Environments p. 155-179. Wiley-Liss, New York.
Huss, V. A. R., Festl, H. & Schleifer, K. H. (1983). Studies on the spectrometric determination of DNA hybridization from renaturation rates. J Syst Appl Microbiol 4, 184-192.
Jancarik, J. & Kim, S. H. (1991). J. Appl. Cryst. 24, 409-411.
Jones, B. E., Grant, W. D., Collins, N. C. & Mwatha, W. E. (1994). Alkaliphiles: diversity and identification. In: Priest F. G., Ramos-Cormenzana A., Tindall, B. J. (Eds) Bacterial diversity and systematic. Plenum Press, New York, pp. 195-229.
Jones, B. E., Grant, W. D., Duckworth, A. W. & Owenson, G. G. (1998). Microbial diversity of soda lakes. Extremophiles 3, 191-200.
Kalisz, M. 1988. Microbial proteinases. Adv. Biochem. Eng/Biotechnol. 36: 1-65.
Koch, C. F., Rainey, F. A. & Stackenbradt, E. (1994). 16S rDNA studies on members of Arthrobacter and Micrococcus: an aid for their future taxonomic restructuring. FEMS Microbiol Lett 123, 16-172.
Kumar, C. G. &Takagi, H. (1999). Microbial alkaline protease: from a bioindustrial viewpoint. Biotechnol Adv 17, 561-594.
Logan, N. A. & Berkeley, C. W. (1984). Identification of Bacillus strains using the API system, J Gen Microbiol 130, 1871-1882.
Martins, R., Davids, W., Abu, W., Levander, F., Radstrom, P. & Hatti-Kaul, R. (2001). Starch-hydrolyzing bacteria from Ethiopian soda lakes. Extremophiles 5, 135-144.
Mota, R. R., Marquez, C., Arahal, D., Mellado, E. & Ventosa, A. (1997). Polyphasic Taxonomy of Nesterenkonia halobia. Int J Syst Bacteriol 47, 1231-1235.
Onishi, H. & Kamekura, M. (1972). Micrococcus halobius sp. n. Int J Syst Bacteriol 22, 233-236.
Outtrup, H. & Boyce, C. O. L. (1990). In Microbial enzymes and biotechnology (ed. W. M. Fogarty & C. T. Kelly), pp. 227-254. New York: Elsevier Science.
Saitou, N. & Nei, M. (1987). The neighbour-joining method: a new method for reconstructing phylogenetic trees. Mol Bio Evol 4, 406-425.
Samal B B, Karan B, Stabinsky Y. Stability of two novel serine proteinases in commercial laundry detergent formulation. Biotechnol Bioeng 1990;35:650-2.
Santos, R. M. D. B., A. A. P. Firmino, C. M. de Sa, and C. R. Felix. 1996. Keratinolytic activity of Aspergillus fumigatus Fresenius. Curr. Microbiol 33: 364-370.
Schleifer, K. H. & Kandler, 0. (1972). Peptidoglycan types of bacterial cell walls and their taxonomic implications. Bacteriol Rev 36, 407-477.
Sharp, R. J., Brown, K. J. & Atkinson, A. (1980). Phenotypic and genotypic characterization of some thermophilic species of Bacillus, J Gen Microbiol 117, 201-210.
Siezen, R. J., and J. A. M. Leunissen. 1997. Subtilases: The superfamily of subtilisin-like serine proteases. Protein Sci 6: 501-523.
Siezen, R. J., de Vos, W. M., Leunissen, J. A. M. & Dijkstra, B. W. (1991). Protein Eng 4, 719-737.
Smibert, R. M. & Krieg N. R. (1994). Phenotypic characterization. p. 611-651.

1. An isolated alkaline protease derived from Nesterenkonia sp. nov. strain deposited at Deutsche Sammlung von Mikroorganismen und Zellkulturen under DSM accession number 15380 on Dec. 20, 2002, having the following characteristics:
molecular weight of 23 kilo Dalton,
melting temperature of about 74° C. at pH 7-10,
calcium independent for activity and stability,
maximal protease activity at pH 10 and retaining 90% of its original activity between pH 6 and 11, and having 60% of its peak activity at pH 12 and being a serine protease, and retains full activity in the presence of 1% (w/v) SDS and 1% (w/v) Triton X-100 at 25° C.

2. An alkaline protease according to claim 1, wherein the protease has a specific activity of at least 1800 U/mg.

3. An alkaline protease according to claim 2, wherein the protease has a specific activity of at least 2200 U/mg.

4. An alkaline protease according to claim 1, wherein the protease exhibits stability against oxidative stress.

5. An alkaline protease according to claim 1 having the amino acid sequence, SEQ, ID. NO. 1, QNPADSPHIGKVFFSTNQGDFVCSANIVASANQSTVATAGHCLHDGNGGQFARNFVFAPAYDYG ESEHGVWAAEELVTSAEWANRGDFEHDYAFAVLETKGGTTVQQQVGTASPIAFNQPRGQYYSAY GYPAAAPFNGQELHSCHGTATNDPMGSSTQGIPCNMTGGSSGGPWFLGQGTGGAQNSVNSYGY TFLPDVMFGPYFGSGAQQNYNYAST.

6. An alkaline protease according to claim 1 encoded by the nucleotide sequence, SEQ. ID. NO. 2 CAG AAT CCG GCG GAC TCC CCG CAC ATA GGC AAG GTC TTC TTC TCC ACC AAC CAG GGC GAC TTC GTC TGC TCC GCC AAC ATC GTG GCC TCG GCG AAC CAG TCC ACG GTG GCC ACC GCG GGG CAC TGC CTG CAC GAC GGA AAC GGC GGC CAG TTC GCA CGC AAC TTC GTC TTC GCC CCT GCC TAC GAC TAC GGC GAG TCC GAG CAC GGC GTG TGG GCC GCA GAA GAG CTG GTG ACC TCC GCC GAG TGG GCG AAC CGC GGC GAC TTC GAG CAT GAC TAC GCC TTC GCG GTC CTC GAG ACC AAG GGC GGC ACC ACC GTG CAG CAG CAG GTG GGG ACG GCG TCG CCG ATC GCC TTC AAC CAG CCG CGC GGC CAG TAC TAC AGC GCC TAC GGC TAC CCG GCC GCC GCG CCC TTC AAC GGC CAG GAG CTC CAC AGC TGC CAC GGC ACC GCC ACG AAC GAC CCG ATG GGC AGC AGC ACT CAG GGC ATC CCG TGC AAC ATG ACC GGC GGC TCC TCC GGC GGC CCC TGG TTc CTC GGT CAG GGG ACC GGC GGT GCC CAG AAC TCT GTG AAC TCC TAC GGG TAC ACC TTC CTG CCG GAC GTG ATG TTC GGG CCG TAC TTC GGC TCC GGG GCA CAG CAG AAC TAC aAC TAC GCc TCC ACA
or a homologue thereof and/or a derivative thereof, wherein the homology is at least 60%, preferably at least 70%, more preferably at least 80%, most preferably 90%.

7. A composition comprising the enzyme of claim 1 and a detergent.

8. A composition comprising the enzyme of claim 1 and a proteinaceous material.

9. A composition according to claim 5, wherein the proteinaceous material comprises keratinous material, such as feathers and/or hair.

10. A composition comprising the enzyme of claim 1 and a bating agent.

11. A composition comprising the enzyme of claim 1 and a silver recovering agent.

12. A composition comprising the enzyme of claim 1 and a food starting material.

13. A method for producing the enzyme of claim 1, said method comprising cultivating a microorganism comprising a nucleotide sequence coding for an alkaline protease under alkaline protease expressing conditions, whereby the nucleotide sequence codes for a homologue and/or a derivative of said alkaline protease, wherein said method comprising providing Nesterenkonia sp. nov. strain, having the following characteristics:
molecular weight of 23 kilo Dalton,
melting temperature of about 74° C. at pH 7-10,
calcium independent for activity and stability,
maximal protease activity at pH 10 and retaining 90% of its original activity between pH 6 and 11, and having 60% of its peak activity at pH 12, and
being a serine protease,
and exposing said strain under conditions where it expresses the said enzyme.

14. A method according to claim 13, wherein the strain is exposed under alkaline conditions.

15. A method according to claim 13, wherein said conditions comprises a growth medium comprising keratinous material, such as feathers and/or hair.

16. A method of degrading proteinaceous material, said method comprising exposing said proteinaceous material to the enzyme of claim 1 under conditions that permit the enzyme to degrade said proteinaceous material.

17. A method according to claim 16, wherein the conditions comprise a pH of 7-12.

18. A method according to claim 16, wherein said proteinaceous material comprises keratinous material, such as feathers and/or hair.

19. A method according to claim 16, wherein said proteinaceous material comprises a soiled wash.

20. A method according to claim 19, wherein said enzyme is present in a combination with a detergent composition.

21. A method according to claim 16, wherein said proteinaceous material comprises leather.

22. A method according to claim 21, wherein said enzyme is present in a combination with a bating composition.

23. A method for recovering silver using a protease methodology, wherein the enzyme of claim 1 is used.

24. A method for food processing using a protease, wherein the enzyme of claim 1 is used.

25. An isolated Nesterenkonia sp. nov. deposited at Deutsche Sammlung von Mikroorganismen und Zellkulturen under DSM accession number 15380 on Dec. 20, 2002.

26. A method of degrading proteinaceous material, said method comprising exposing said proteinaceous material to the strain Nesterenkonia sp. nov. of claim 25 under conditions that permit the strain to produce an alkaline protease that degrades said proteinaceous material.

27. A method of producing an alkaline protease having the following characteristics:
molecular weight of 23 kilodalton,
melting temperature of about 74° C. at pH 7- 10,
calcium independent for activity and stability,
maximal protease activity at pH 10 and retaining 90% of its original activity between pH 6 and 11, and having 60% of its peak activity at pH 12, and
being a serine protease,
said method comprising
providing the strain of claim 26,
and culturing the said strain under conditions where the alkaline protease is expressed.

28. A method according to claim 27, wherein the protease is purified.

29. (canceled)

30. (canceled)

31. (canceled)

32. (canceled)

33. (canceled)

34. (canceled)


SEQUENCE LISTING (8 sequences)

SEQ ID NO: 1 - protein, 216 aa, Nesterenkonia sp.
QNPADSPHIGKVFFSTNQGDFVCSANIVASANQSTVATAGHCLHDGNGGQFARNFVFAPAYDYGESEHGVWAAEELVTSAEWANRGDFEHDYAFAVLETKGGTTVQQQVGTASPIAFNQPRGQYYSAYGYPAAAPFNGQELHSCHGTATNDPMGSSTQGIPCNMTGGSSGGPWFLGQGTGGAQNSVNSYGYTFLPDVMFGPYFGSGAQQNYNYAST

SEQ ID NO: 2 - DNA, 648 bp, Nesterenkonia sp.
cagaatccggcggactccccgcacataggcaaggtcttcttctccaccaaccagggcgacttcgtctgctccgccaacatcgtggcctcggcgaaccagtccacggtggccaccgcggggcactgcctgcacgacggaaacggcggccagttcgcacgcaacttcgtcttcgcccctgcctacgactacggcgagtccgagcacggcgtgtgggccgcagaagagctggtgacctccgccgagtgggcgaaccgcggcgacttcgagcatgactacgccttcgcggtcctcgagaccaagggcggcaccaccgtgcagcagcaggtggggacggcgtcgccgatcgccttcaaccagccgcgcggccagtactacagcgcctacggctacccggccgccgcgcccttcaacggccaggagctccacagctgccacggcaccgccacgaacgacccgatgggcagcagcactcagggcatcccgtgcaacatgaccggcggctcctccggcggcccctggttcctcggtcaggggaccggcggtgcccagaactctgtgaactcctacgggtacaccttcctgccggacgtgatgttcgggccgtacttcggctccggggcacagcagaactacaactacgcctccaca

SEQ ID NO: 3 - protein, 30 aa, Unknown Organism
  Description of Unknown Organism Insulin B-chain sequence
FVNQHLCGSHLVEALYLVCGERGFFYTPKA

SEQ ID NO: 4 - DNA, 20 bp, Artificial Sequence
  Description of Artificial Sequence Synthetic primer
agagtttgatcctggctcag

SEQ ID NO: 5 - DNA, 19 bp, Artificial Sequence
  Description of Artificial Sequence Synthetic primer
ggttaccttgttacgactt

SEQ ID NO: 6 - protein, 4 aa, Artificial Sequence
  Description of Artificial Sequence Synthetic peptide
LLVY

SEQ ID NO: 7 - protein, 4 aa, Artificial Sequence
  Description of Artificial Sequence Synthetic peptide
AAPF

SEQ ID NO: 8 - protein, 4 aa, Artificial Sequence
  Description of Artificial Sequence Synthetic peptide
AAPL